(19) United States
(12) Patent Application Publication
(10) Pub. No.: US 2026/0112118 A1
(43) Pub. Date: Apr. 23, 2026

(54) STANDARDIZED COORDINATE SYSTEMS FOR MAPPING THREE-DIMENSIONAL STRUCTURES

(71) Applicant: THE REGENTS OF THE UNIVERSITY OF CALIFORNIA, Oakland, CA (US)

(72) Inventors: Elliot McVeigh, San Diego, CA (US); Noah Mehringer, San Diego, CA (US)

(21) Appl. No.: 19/213,841

(22) Filed: May 20, 2025

Related U.S. Application Data
(60) Provisional application No. 63/711,076, filed on Oct. 23, 2024.

(51) Int. Cl. G06T 17/20; G06T 7/00; G06T 7/12; G06T 7/13; G06V 10/44; G16H 30/20; G16H 30/40

(52) U.S. Cl. CPC G06T 17/20; G06T 7/0012; G06T 7/12; G06T 7/13; G06V 10/44; G16H 30/20; G16H 30/40; G06T 2207/30048

(57) ABSTRACT

The present technology relates to standardized coordinate systems for mapping three-dimensional structures. An exemplary method includes: obtaining imaging data including a representation of a left atrium of a subject; identifying anatomical features represented in the imaging data, the anatomical features including pulmonary veins that connect to the left atrium; generating a 3D surface mesh corresponding to the left atrium; determining a reference point on the 3D surface mesh based on locations of junctions of the pulmonary veins with the left atrium; establishing a reference axis based on the reference point; determining, on the 3D surface mesh and based on the reference axis, lines of longitude; determining, on the 3D surface mesh, lines of latitude along the lines of longitude; assigning coordinate values to points on the 3D surface mesh based on the lines of longitude and latitude; and generating a 2D map of the left atrium using the coordinate values.

### CROSS-REFERENCE TO RELATED APPLICATION(S)

This application claims priority to the provisional application with Ser. No. 63/711,076, titled “COORDINATE SYSTEM FOR THE LEFT ATRIUM OF THE HEART,” filed Oct. 23, 2024. The entire contents of the above noted provisional application are incorporated by reference as part of the disclosure of this document.

### STATEMENT REGARDING FEDERALLY SPONSORED RESEARCH

This invention was made with government support under R01 HL160024 awarded by the National Institutes of Health. The U.S. government has certain rights in the invention.

### TECHNICAL FIELD

The present technology relates to imaging analysis, and particularly to methods and systems for mapping of a three-dimensional representation of a structure to a two-dimensional representation.

### BACKGROUND

Three-dimensional (3D) imaging technologies have become increasingly prevalent across various fields, including medicine, engineering, geology, and architecture. These imaging modalities generate detailed volumetric data that can represent complex structures with high fidelity. The analysis of such 3D structures often involves examining surface properties, identifying patterns, and making comparisons between different instances of similar structures or tracking changes in a single structure over time. Traditional approaches to handling 3D structural data have included direct 3D visualization techniques, data sampling at predefined locations, and various projection methods to represent 3D information in more accessible formats.

### SUMMARY

An aspect of the present document relates to a computer-implemented method. In some embodiments, the method includes: obtaining imaging data including a representation of a left atrium of a subject; identifying anatomical features represented in the imaging data, the anatomical features including pulmonary veins that connect to the left atrium; generating a three-dimensional (3D) surface mesh corresponding to the left atrium; determining a reference point on the 3D surface mesh based on locations of junctions of the pulmonary veins with the left atrium; establishing a reference axis based on the reference point; determining, on the 3D surface mesh and based on the reference axis, lines of longitude that traverse the reference point; determining, on the 3D surface mesh, lines of latitude as isolines of equal normalized distance along the lines of longitude; assigning coordinate values to points on the 3D surface mesh based on the lines of longitude and latitude; and generating a two-dimensional (2D) map of the left atrium using the coordinate values, wherein the 2D map enables a visualization or comparison of a physiological parameter across different regions of the left atrium for clinical management for the subject or a subject group that includes at least another subject other than the subject.

Another aspect of the present document relates to a computer-implemented method. In some embodiments, the method includes: obtaining imaging data including a representation of a three-dimensional (3D) target component; identifying, by segmenting the imaging data, the target component and feature regions associated with the target component; generating a 3D surface mesh corresponding to the target component; establishing, based on the feature regions, a reference axis associated with the 3D surface mesh; determining, on the 3D surface mesh and based on the reference axis, lines of longitude; determining, on the 3D surface mesh, lines of latitude transverse to the lines of longitude; assigning coordinate values to points on the 3D surface mesh based on the lines of longitude and latitude; and generating a two-dimensional (2D) representation of the target component using the coordinate values

A further aspect of the present document relates to a system. In some embodiments, the method includes: at least one processor; and memory with instructions stored thereon, wherein the instructions upon execution by the at least one processor, cause the at least one processor to perform a method disclosed herein.

A still further aspect of the present document relates to one or more non-transitory computer readable program storage media. In some embodiments, the one or more non-transitory computer readable program storage media having code stored thereon, the code, when executed by at least one processor, causing the at least one processor to implement a method disclosed herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fees.

FIGS. 1A and 1B show an American Heart Association (AHA) formulation of a 2D coordinate system for the left ventricle.

FIG. 2 shows left atrium standardized unfold map (LA-SUM) unwrapping of the left atrium onto a flat disc.

FIG. 3 shows standardized 2D atrial mapping of the left and right atria.

FIGS. 4A-4C show Universal Atrial Coordinates of a left atrium (LA) represented on a 3D geometry and on a flat square.

FIGS. 5A and 5B show a coordinate system framework for a Left Atrial Positioning System (LAPS), according to embodiments of the present technology.

FIG. 6 shows a Robinson projection map of the world's land and oceans.

FIG. 7 illustrates a hypothetical angular coordinate represented on the contour of a real atrial wall.

FIG. 8 shows LAPS coordinates displayed on a gallery of LA geometries, according to embodiments of the present technology.

FIG. 9 shows LA differences in various test groups.

FIG. 10 shows an LA long axis defined using an arbitrary LA geometry, shown in a left-oblique view with a transparent surface, according to embodiments of the present technology.

FIG. 11 shows a roof line, prime meridian, and an example line of latitude (the “equator”), according to embodiments of the present technology.

FIG. 12 illustrates determination of Left Atrial Positioning System (LAPS) coordinates for a single target node, according to embodiments of the present technology.

FIGS. 13A-13D show LAPS coordinates used to automatically divide and display the LA body into subregions, according to embodiments of the present technology.

FIGS. 14A-14D illustrate a gallery of exemplary world map projections.

FIG. 15 demonstrates a gallery of scalar strain data from a left atrium, plotted using various cartographic projections, according to embodiments of the present technology.

FIG. 16 demonstrates the Peirce-Quincuncial projection algorithm, implemented in MATLAB.

FIG. 17 shows a one-way error validation process, according to embodiments of the present technology.

FIG. 18 shows a 3D LA shape projected onto a Peirce Quincuncial square, according to embodiments of the present technology.

FIG. 19 shows one-way transfer errors averaged across scans and projected onto a Peirce-Quincuncial square, according to embodiments of the present technology.

FIG. 20 shows a visualization of LA wall labels with respect to relevant anatomy, according to embodiments of the present technology.

FIG. 21 shows subdivisions of the LA walls, according to embodiments of the present technology.

FIGS. 22A and 22B illustrate LA Bullseye plots for 54 and 96 segments, respectively.

FIG. 23 shows Left Atrial Positioning System (LAPS), according to embodiments of the present technology.

FIGS. 24A-24C show Peirce-Quincuncial projection warps the LA surface similar to its intended use in global cartography, according to embodiments of the present technology.

FIG. 25A illustrates a schematic representation of atrial fibrillation (AF).

FIG. 25B depicts a schematic view of an ablation-induced scar in the left atrium, as identified using magnetic resonance imaging (MRI).

FIGS. 26A-26D show an exemplary implementation of the standardized coordinate system, according to embodiments of the present disclosure.

FIG. 27 shows the change in RSct strain in ablated versus non-ablated regions, obtained from the exemplary implementation as illustrated in FIGS. 26A-26D.

FIG. 28 illustrates the functional consequences of ablation on regional strain using net RSCT maps obtained from the exemplary implementation as illustrated in FIGS. 26A-26D.

FIG. 29 shows the flowchart of a process for left atrium mapping, according to embodiments of the present disclosure.

FIG. 30 shows the flowchart of a process for mapping a three-dimensional target component, according to embodiments of the present disclosure.

FIG. 31A shows a block diagram of an example system, according to embodiments of the present technology.

FIG. 31B shows a block diagram of an exemplary processing device, according to embodiments of the present technology.

### DETAILED DESCRIPTION

The present technology relates to imaging analysis, and particularly to methods and systems for generating standardized coordinate systems to enable mapping of a three-dimensional representation of a structure to a two-dimensional representation. The present technology utilizes an algorithm that receives measured data corresponding to automatically convert a 3-dimensional (3D) surface mesh of a structure (e.g., a left atrium) and automatically converts the data into a standard 2-dimensional space. As an exemplary implementation, the present technology provides a Left Atrial Positioning System (LAPS) for cardiac imaging applications.

### I. INTRODUCTION

Cardiovascular imaging and electrophysiology applications in the left atrium (LA) are increasingly requiring the integration of multi-modality datasets to guide treatment and monitor progress in individual patients. Registration algorithms are traditional tools to spatially correlate 3D cardiovascular datasets; however, they become less accurate in maintaining landmark correspondence and more computationally expensive when the difference between two registered geometries is larger. This presents a challenge when studying the LA over long periods of time because individual patients often experience significant LA mechanical remodeling in response to disease and treatment.

In addition, cohort studies of diseases affecting the LA involve comparisons of 3D endocardial surface data across patients which are difficult because the shape of the LA is irregular and has a high degree of interpatient variation in gross morphology, pulmonary vein (PV) arrangement, and left atrial appendage (LAA) variety.

The present technology addresses a need to perform automatic comparisons of 3D LA imaging datasets in patients with atrial fibrillation to measure responses to treatment and create statistical models of the different patient sub-populations. Compared to existing technology, the present technology enables pointwise comparisons between LA images having significantly different shapes—being taken from different patients or after significant LA shape remodeling—and doing so automatically from easily identified anatomic landmarks.

Cardiovascular coordinate reference systems permit the integration of information from multiple images by parameterizing the shape of the cardiac chamber—which is embedded in Cartesian space from medical imaging—with new coordinates derived from anatomic landmarks. For example, the popular “bullseye” coordinate system for the left ventricle (LV) describes local position with a pseudo-cylindrical coordinate system calculated with respect to the left ventricle longitudinal axis. This system is used in clinical research and by major medical imaging vendors to conduct studies on LV function, perfusion, electrophysiology, etc.; however, coordinate reference systems for the LA have not emerged until the past decade.

Previous approaches have assigned points on an LA mesh to fall within a regional boundary on the mesh surface—defined either on an atlas mesh or on individual subjects. The target mesh is then unwrapped into 2D geometric shapes using standardized unfold mapping, conformal, or quasi-conformal flattening techniques. These methods serve the purpose of 2D visualization and in some cases automatic subregion labeling. However, they are not optimal for pointwise comparison of datasets because each point is assigned a 2D location derived from the image-specific transformation rather than with respect to common anatomy. In this respect, the unfolding techniques are more akin to a registration algorithm than a coordinate system.

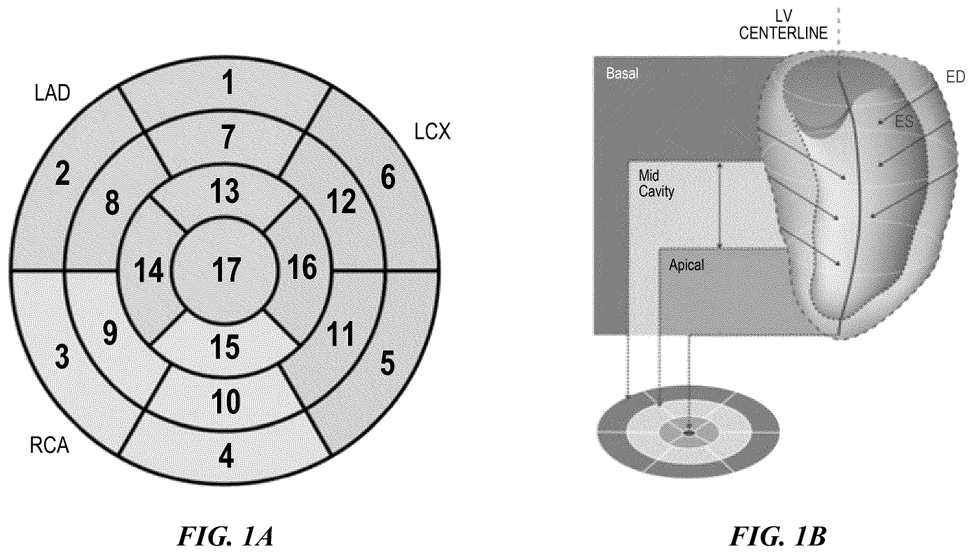

There is already a widely adopted coordinate system in cardiovascular medicine; the American Heart Association (AHA) endorsed a pseudo-cylindrical system for the left ventricle (LV) in 2002. The system characterizes the LV using a longitudinal axis running from the apex to the base of the LV through the center of the luminal space and a second, angular coordinate emanating from the axis like the spokes of a wheel. As a result, the LV wall can be subdivided into regions which have been shown to be consistent across many LV shape variations. The so-called AHA “bullseye” plot represents the LV wall in 17 segments, allowing for easy visualization of data across the entire surface (FIGS. 1A and 1B).

As illustrated in FIGS. 1A and 1B, the AHA formulation of a 2D coordinate system for the left ventricle. FIG. 1A illustrates the LV bullseye plot with 17 anatomical segments linked to the territories of the 3 major coronary arteries. FIG. 1B illustrates the ventricle is divided into apical, middle, and basal regions for division into the 17 segments.

The AHA bullseye demonstrates the useful properties of coordinate reference systems. The system allows for integration of data from different imaging modalities—for example, voltages from electroanatomic mapping catheters with scar tissue from late gadolinium-enhanced MRI (LGE-MRI) to treat ventricular arrhythmias and PET/CT fusion to localize infarctions. The projection of 3D and 4D left ventricle data onto a 2D space permits the reader to visualize all pertinent data on a screen or medical chart. This is a seemingly banal but foundational element for smooth clinical adoption: new cardiovascular data representations need to be presented in a way aligned with procedural workflows. The AHA segments also link locations on the LV wall to relevant anatomical structures—the coronary arteries. Segments affected by decreased myocardial perfusion are linked to a coronary artery, so the perfusion plots can direct needed intervention to the appropriate vessel(s).

Coordinate systems for the left atrium exist, but none have been widely adopted like the LV system. Perhaps the clinical usefulness of an LA system is not yet evident enough to demand standardization. LV segments were modality-specific for decades before being united under the AHA. However, the electrophysiology industry is driving demand for complex atrial data representation, including the integration of electroanatomic maps with CT anatomy and LGE-MRI scar. To meet these needs, several research groups have developed systems to parameterize positions on the left atrium.

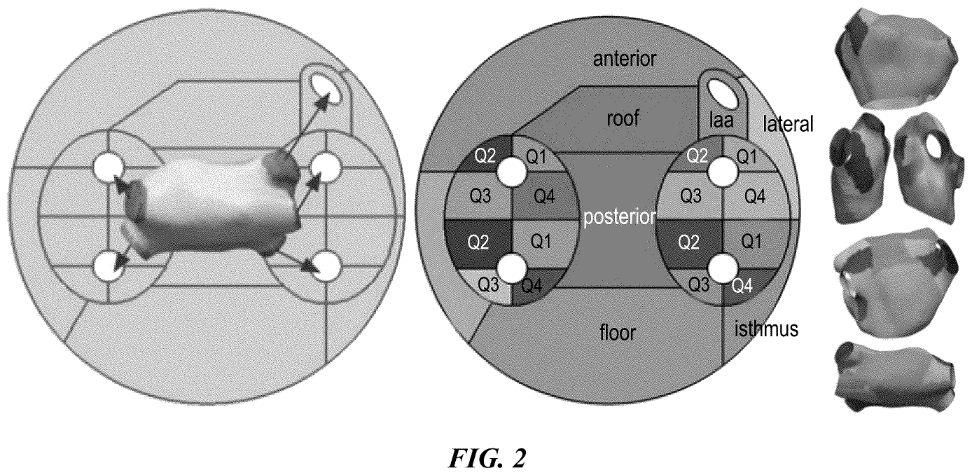

A prior study has illustrated an LA standardized unfold map (LA-SUM) methods, which created a shape atlas created from 18 subjects and unwrapped the resulting surface onto a 2D disc. The projection constrained the mitral valve to the outer disc and the PVs and LAA to geometric shapes within the disc. Twenty-four subregions are defined in the atlas (FIG. 2). New LA datasets may be non-rigidly registered to the atlas before undergoing the same unwrapping process. FIG. 2 shows LA-SUM unwrapping of the left atrium onto a flat disc.

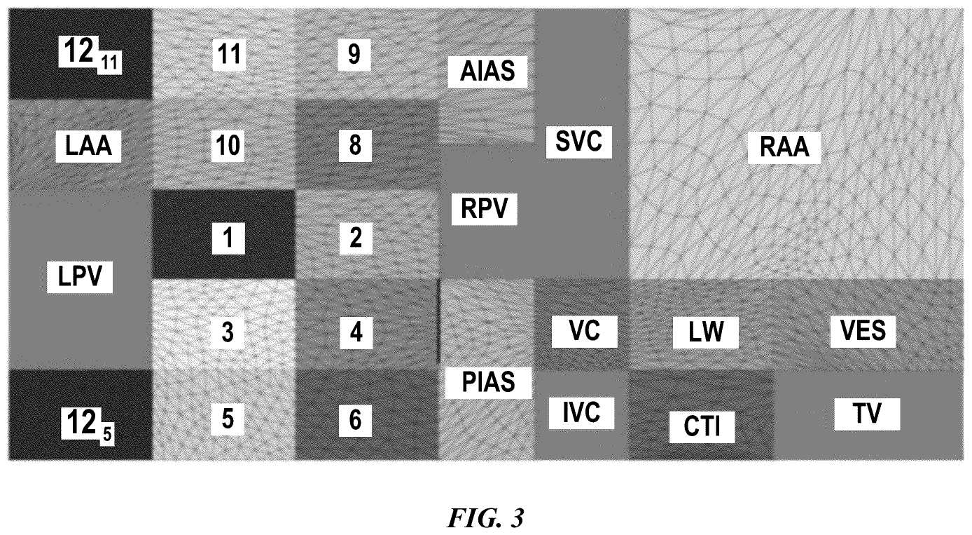

Another prior study has disclosed a similar technique which requires the 3D LA mesh input be preemptively divided into 19 subregions, and each region in sequence is mapped to a square for display as illustrated in FIG. 3 showing standardized 2D atrial mapping of the left and right atrium.

These two systems require non-trivial pre-processing (the creation of an atlas or subregion assignment). Additionally, they are designed chiefly for regional comparisons of data between images and are not optimized for transferring individual data points between shapes. For example, if a voltage data point was collected during an electrophysiology study and mapped to the posterior wall on the LA-SUM disc, it may be desirable but challenging to map that data point back to 3D during a follow-up electrophysiology study. The 2D mapping cannot be inverted because the mapping is dependent on the non-rigid deformation of a particular image. The LA shape can change drastically throughout the cardiac cycle, and especially over longer periods of time in patients with heart disease. A point-to-point mapping needs to rely on unchanging features of the LA anatomy.

The Universal Atrial Coordinate (UAC) system, by contrast, assigns unique coordinates to every point on an LA mesh and likewise for the right atrium (RA). The coordinates are derived by solving the Laplace equations across the atrial surface, with boundary conditions defined by the mitral valve (MV) and user-specified selection of the fossa ovalis, superior PVs, and LAA. The UAC was an improvement over the previous unfolding techniques because—like for the LV—individual points may be assigned a coordinate location in reference to anatomic landmarks. The point transfer principle was validated by comparing the transfer of electroanatomic data to 3D shapes from LGE-MRI against a clinically used nonrigid registration algorithm. Dividing the LA into regions was a trivial next step, and the group recently published work dividing the UAC into 5 anatomic subregions. The UAC is an optimal LA coordinate system to date in terms of serving many functions and showing promise for further adoption.

For example, in a prior study regarding the Universal Atrial Coordinate (UAC) system, each point on the surface of an LA mesh is assigned a septal-lateral coordinate and a posterior-anterior coordinate, allowing for pointwise transfer of scalar and vector data. The coordinates are calculated by first defining boundary lines on the surface mesh using the PVs, fossa ovalis, and mitral valve. These are reliably fixed landmarks present in all atria. The alpha coordinate is bounded by 0 at the septal wall and 1 at the lateral wall. The Laplace equations are solved across the LA surface with those boundary conditions. The beta coordinate is similarly defined by beginning at the posterior mitral annulus and traveling over the top of the LA to the anterior mitral annulus.

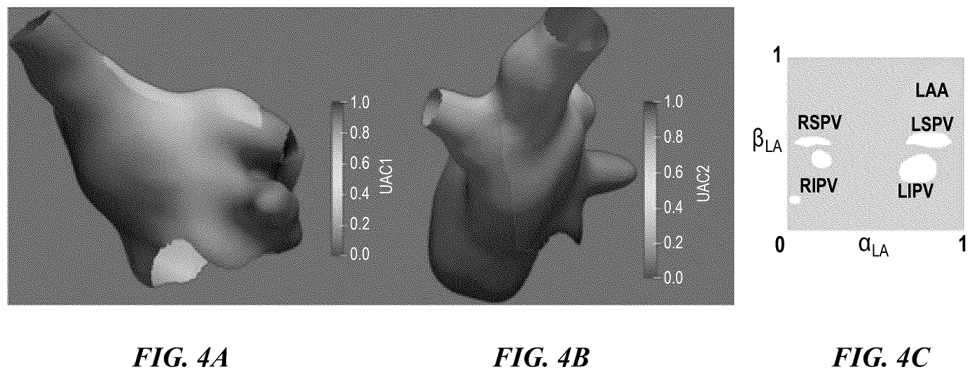

FIGS. 4A-4C show Universal Atrial Coordinates of a left atrium represented on a 3D geometry and on a flat square. FIG. 4A shows the septal-lateral coordinate, alpha (“UAC1”). FIG. 4B shows the posterior-anterior coordinate, beta (“UAC2”). There is a discontinuity in this coordinate visible at the septal and lateral walls. FIG. 4C shows four pulmonary veins, the left atrial appendage, and the fossa ovalis of the left atrium plotted in 2D Universal Atrial Coordinates.

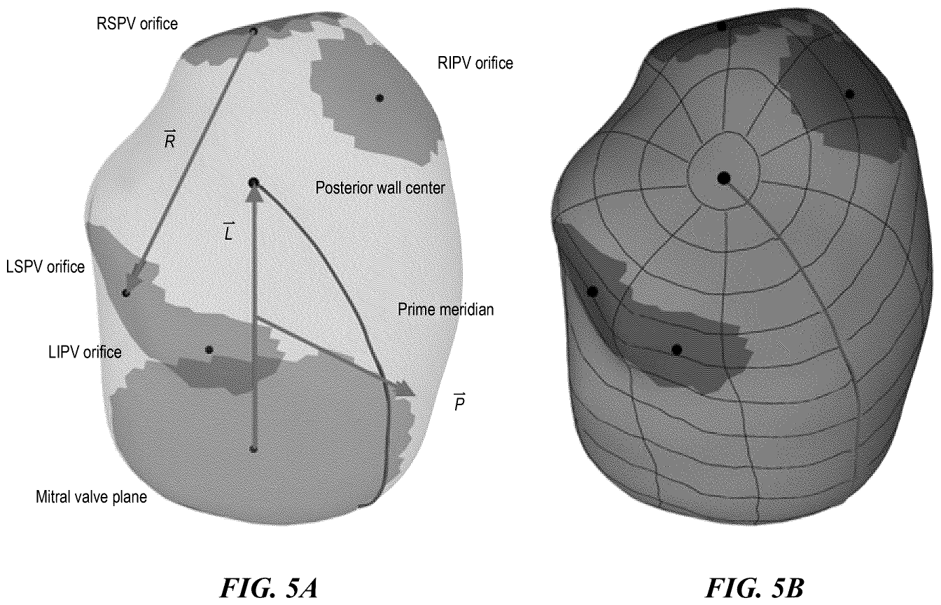

The UAC leaves various challenges unsolved. Prior studies show that key UAC landmarks are not consistently located from all image sets. In the majority of 4DCT studies the RA cannot be easily segmented due to lack of x-ray contrast material in the RA chamber; hence, the fossa ovalis cannot be accurately determined. Many subjects were missing an LAA entirely, since the LAA is increasingly being occluded or removed by cardiac interventions. Prior studies also found that the takeoff of the LAA from the atrial body has significant posterior-anterior variation across patients (FIGS. 5A and 5B). Accordingly, the UAC may need the manual selection of a few landmark points, including the fossa ovalis, which are not apparent in many LA segmentations—the group labeled the fossa ovalis using the overlap from a right atrium segmentation. These are inconveniences which may scale into headaches when processing datasets in the hundreds or thousands of images. Error in the selection of these landmarks visibly distorts the UAC coordinates. In addition, the manual selection of even a few landmark points becomes unrealistically laborious to process hundreds or thousands of datasets, which the present technology addresses. The present technology provides a system for parameterizing the LA which is better suited for large studies of LA surface data that can be automatically determined.

Additionally, it was also demonstrated that the solution to the Laplace equation is an inaccurate measure of normalized distance since the linearity of the coordinates is dependent on the width of the domain. In a prior study, a comparison between Bayer and Roney's Universal Ventricular Coordinates and a ventricular coordinate system named “Cobiveco” shows that the UVC produced a mean error of 7 mm when transferring data from one LV geometry to another, while the error of the Cobiveco model was 1.5 mm. Laplace solution point transfer errors are likely to be more severe in the LA, which has a less uniform geometry than the LV.

Beyond the challenges of spatial registration across different patients, additional difficulties exist in tracking points on the left atrium throughout the cardiac cycle. To proceed with the calculation and application of local strain from 4DCT, challenges exist in dealing with complex, four-dimensional data. The shape of the left atrium can change quite dramatically throughout the cardiac cycle as it contracts, and yet we want to track scalar or vector data on its surface. The images collected from 4DCT provide snapshots of the heart in different poses at a frame rate of 10-20 Hz. Practically, the resulting dataset is a series of 3D shapes. The temporo-spatial resolution of 4DCT is not sufficient to track local features on the atrial wall (and there are not many features to track), so the series of shapes are correlated in time, but individual fiducial points are not tracked. Performing computations between shapes without direct point correspondence becomes somewhat nonsensical—like adding a square to a circle—without the use of a common reference system. Without a standard system to index locations of elements on the LA, the various shapes are unmoored in time, and it is unclear where a point on the surface travels to in the next timeframe. An exemplary implementation may include measuring the change in local LA strain over long periods of time (months to years) and create statistical atlases of large patient cohorts. This needs a common coordinate system to compare data between 4DCT scans of the same patient and between different patients.

The present technology provides standardized coordinate systems to enable mapping of three-dimensional representations of structures to two-dimensional representations. For illustration purposes and not intended to be limiting, an exemplary implementation as a Left Atrial Positioning System (LAPS) is described. The system allows fully automated conversion of three-dimensional left atrium surface meshes into a standard two-dimensional space. The LA endocardial surface—excluding the LAA and PVs—is parameterized with surface contours analogous to lines of latitude and longitude. The coordinate assignment may be implemented in MATLAB on LA volume segmentations from 4-dimensional computed tomography (4DCT). Pointwise data transfer accuracy with LAPS between scans of the same subject taken months apart and between different subjects was evaluated against synthetic ground truth models. In exemplary implementations, the LAPS coordinates were used to automatically divide the LA into 24 subregions for display on a standardized square plot that covers the LA body. The subregions from LAPS coordinates were used to compare regional responses to surgical treatment in the same subjects over time. The subregions from LAPS coordinates were applied to a large patient cohort to generate a statistical model of regional LA characteristics for the patient population. LAPS coordinates were used to co-register left atrium surface data from one imaging study to another in the same subject taken months and years apart. Technical benefits of the system include: (1) full automation from left atrium segmentation; (2) high accuracy when transferring points between left atrium shapes; (3) normalized coordinates that span the same range regardless of atrium size; (4) robustness to variable LA body shapes; (5) definitions from easily identifiable LA anatomy; and (6) flexible visualization where data may be easily represented in many forms depending on the application.

### II. COORDINATE SYSTEM

### II.A. Theory of Construction

The LV bullseye system reduces the dimension of a 3D geometry by computing a unique angle and radius for each point on the endocardial surface. However, the irregular shape of the LA does not lend itself to angular coordinates; it is often not convex and not symmetrical about any axis which can lead to singularities in the mapping of angles to the surface. Instead, normalized distance computed across the LA surface is appropriate to ensure the coordinate space represents an injective function for the shape. Excluding the PV sleeves and LAA surfaces will help reduce local coordinate distortions.

When defining an anatomic coordinate system, it is useful to choose landmarks which are consistently located with respect to each other and consistent across the population. The most consistent LA anatomic features across patients are the location of the mitral valve and the presence of pulmonary veins at the posterior wall of the atrium (FIG. 5A). Although the number and precise structure of the veins are variable, they reliably flank the posterior wall, a wall which itself is approximately opposite the mitral valve. The left atrial appendage is an unreliable landmark, since an increasing number of AF patients are undergoing LAA exclusion to reduce their risk of stroke. For this reason, and because the LAA itself is highly variable in shape and prone to causing coordinate distortion, the decision was made early on to exclude the LAA from a coordinate system.

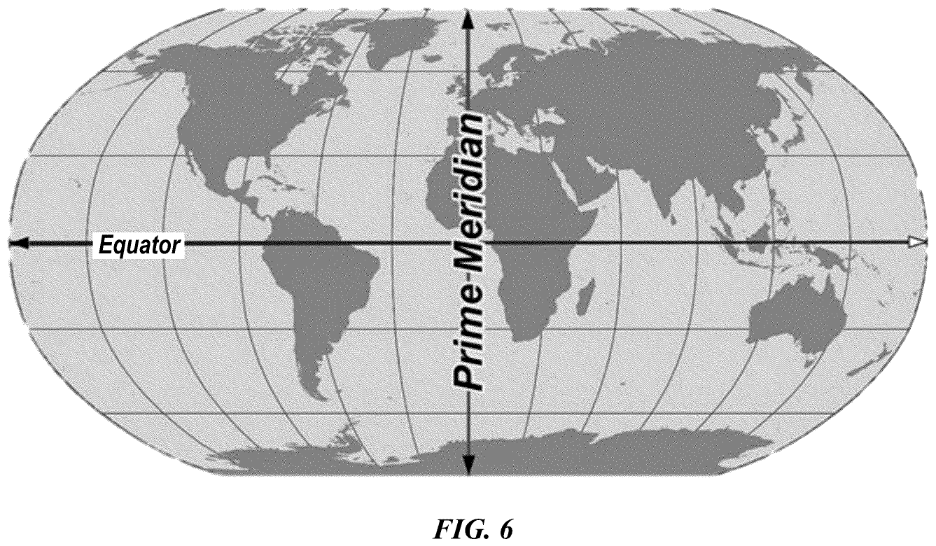

Inspiration was drawn from the world's first coordinate system: latitude and longitude. When referencing locations on Earth, the planet is approximated as an ellipsoid so that great circles may be mathematically defined on its surface. The lines of latitude and longitude can be indexed in degrees, minutes, and seconds to accurately locate geographic features. FIG. 6 shows a Robinson projection map of the world's land and oceans. The Equator is a circumferential line calculated to bisect the globe into northern and southern hemispheres. The Prime Meridian is a single line of longitude that serves as the reference point for other meridians.

However, the approximation of an ellipsoid creates many problems in cartography, since the Earth's surface in fact has many irregularities, mountains, and trenches for which an ellipsoid approximation may err by hundreds of meters. For this reason, many political entities (even individual counties within the United States, for example) define their own unique ellipsoid which works best for their region of the world.

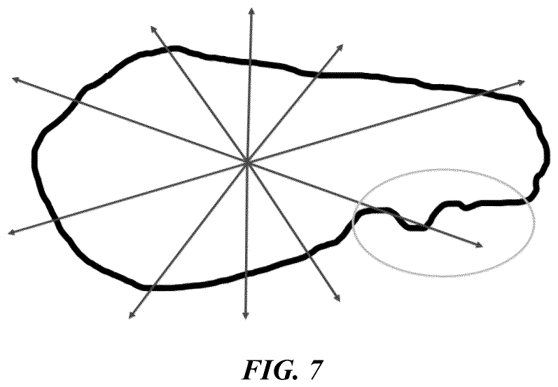

The left atrium is a closed, roughly convex shape resembling a sphere, especially in its most engorged position, but such a drastic spherical approximation is not necessary to map the human left atrium. Unlike the Earth's surface, the LA endocardial surface can be fully modelled from segmented medical images. Although the generation of a discrete mesh from an image with limited resolution inherently approximates some very local aspects of the shape, the form of the LA is generally well-preserved in a high-quality segmentation. Rather than defining lines of latitude and longitude with the great discs of a sphere or ellipsoid, contours may be traced directly on the surface mesh. The key advantage of linking the coordinates to the surface of LA—rather than choosing an angular coordinate (FIG. 7), like in the AHA LV system—is that the coordinate system may still be one-to-one even if the shape breaks convex assumptions. A surface coordinate also guarantees that the coordinates can be linearly distributed.

FIG. 7 illustrates a hypothetical angular coordinate represented on the contour of a real atrial wall. In the region marked by the circle the shape is not convex, so multiple points are assigned the same coordinate value. The intersections of the coordinate lines with the contour are not linearly distributed but depend on the local geometry.

In some embodiments, LAPS is formulated by first defining an axis passing through the mitral valve (MV) center and posterior wall centers (defined as the center of mass of the pulmonary vein (PV) orifices), respectively, then computing meridians on the surface—connecting the poles of the axis—and finally computing lines of latitude along the equal values of the meridians. A “prime meridian” is the line of longitude which travels through the posterior wall center and descends the posterior side of the atrium—dividing the LA into right and left halves; it serves as the zero-reference for longitude coordinates. It should be noted that true “latitude” and “longitude” are well-defined terms respecting a mathematical ellipsoid; however, the coordinates in LAPS are computed on an arbitrary surface, and so the cartographic terminology is used here as a helpful analogy.

### II.B. Data Collection

Contrast-enhanced 4DCT datasets were collected retrospectively under IRB approval from a single center. All subjects received their 4DCT scan in preparation for an atrial fibrillation ablation. Data was assembled into 2 test groups. Group 1—used for intrasubject LAPS validation—constituted any patient who had undergone two 4DCT scans from 2018 through 2022 with an atrial fibrillation ablation in between. These numbered 15 subjects and 30 image datasets. To create Group 2—used for intersubject LAPS validation—an additional 15 patients' scans from the last 3 months of 2022 were collected. See Tables 1 and 2. These new scans were combined with the post-ablation Group 1 scans and shuffled into two cohorts, A and B. Each subject in Group 2, cohort A was paired with a subject in Group 2, cohort B to test intersubject LAPS accuracy. To retain randomness in subject-pairing, these subjects were not matched by any metric. Patient records were pulled for demographic information and measures of body size.

### II.C. Subject Characteristics

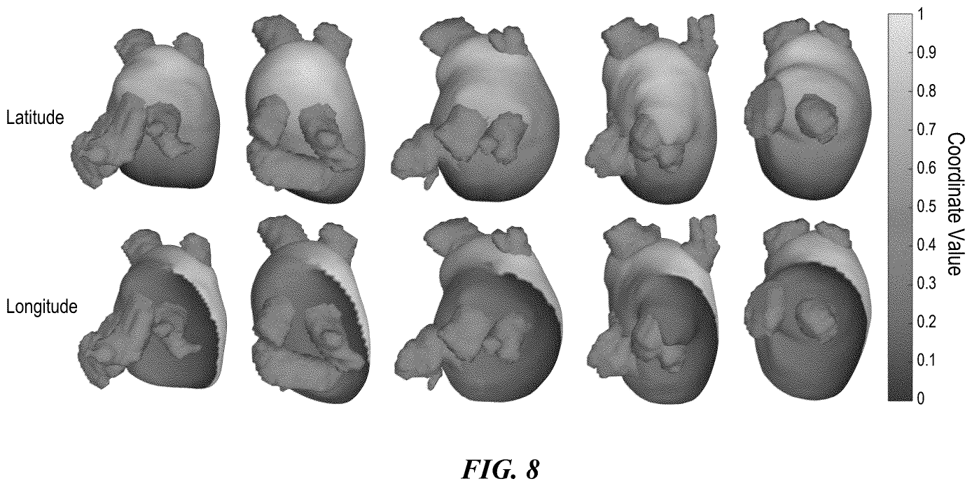

The atria were notably enlarged, having a mean LA volume index (LAVi) of 73.1 and a surface area of 150 cm² (Table 1). Subjects had a mean age of 67 and all were diagnosed with atrial fibrillation. The dataset of 45 scans comprised a variety of LA sizes and shapes, including configurations with 3 PVs and missing LAA (FIG. 8). LAPS coordinates were successfully computed on the surface of each reservoir phase volume. Runtime per subject was 1-2 minutes on an 8-core Intel i7 CPU.

TABLE 1
SUBJECT CHARACTERISTICS

| | Group 1 | Group 1 | Group 2 | Group 2 | |
|---|---|---|---|---|---|
| | Pre-Ablation | Post-Ablation | Cohort A | Cohort B | All Scans |
| Characteristic | (n = 15) | (n = 15) | (n = 15) | (n = 15) | (n = 45) |
| Age | 67 | 69 | 67 | 65 | 67 |
| | (55-82) | (59-83) | (38-82) | (35-83) | (35-83) |
| Sex | 10/15 | | 6/15 | 8/15 | 14/30 |
| (Female/Total) | | | | | |
| BSA | 2.04 | 2.03 | 2.14 | 1.97 | 2.05 |
| | (1.65-2.59) | (1.59-2.57) | (1.81-2.57) | (1.59-2.23) | (1.59-2.59) |
| LAV, Reservoir | 157.3 | 151.3 | 144.6 | 139.4 | 147.1 |
| (mL) | (116.9-213.4) | (105.5-205.3) | (94.8-240.7) | (65.6-186.6) | (65.6-240.7) |
| LAVi | 78.3 | 76.4 | 68.7 | 72.4 | 73.1 |
| | (53.2-119.2) | (46.1-113.4) | (41.2-122.2) | (29.4-103.9) | (29.4-122.2) |
| LA Surface Area | 155.0 | 149.9 | 149.4 | 145.6 | 150.0 |
| (cm²) | (126.3-183.5) | (119.0-170.6) | (118.4-220.7) | (91.8-180.0) | (91.8-220.7) |

Numeric data in Table 1 is reported as a mean and a range. BSA=body surface area, LAV=left atrium volume, LAVi=left atrium volume index.

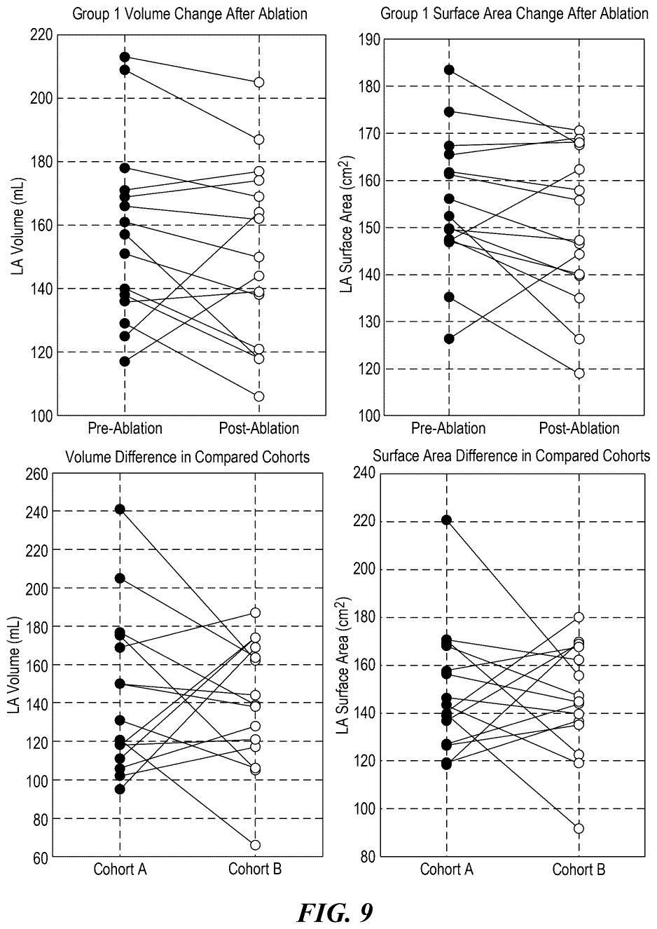

In Group 1, where each subject received an ablation for atrial fibrillation between 4DCT scans, the average time between the first and second scan was 21 months (range 4-38). This allowed time for mechanical remodeling of the LA in response to the ablation or due to the progression of AF or heart failure. The degree of remodeling in response to ablation in Group 1 ranged from a 25% decrease in volume to a 30% increase, although the mean volume and surface area as a group only decreased 3% (FIG. 9, Table 1).

Group 2 had larger variation in LA volume and surface area differences, owing to the random pairing of patients in cohorts A and B. The volume difference from A to B ranged −46% to +78% and surface area from −34% to +43% (FIG. 9).

FIG. 9 shows LA differences in each test group. On average, Group 1 experienced a small volume and surface area decrease after ablation. For the intersubject comparisons in Group 2, some subject pairs were of approximately equal size, whereas others had drastic differences.

### II.D. Mesh Generation

Left heart volumes at every time frame of the 4DCT images were automatically segmented into 9 chambers (LV, LA, LAA, four PVs, ascending aorta, and left ventricular outflow tract) using a U-Net semantic segmentation network built and trained in-house with a database of expert segmentations. Segmentations were visually inspected for quality and manually edited as needed to remove artifacts, such as from metal leads and LAA exclusion devices. The planes separating the LA body from the LV (the mitral valve plane), LAA, and PVs (PV planes) were automatically identified by dilating each labeled volume and storing the locations where the labels overlapped. The LA volumes were converted into triangular surface meshes with a resolution of 2 mm. The mesh nodes preserved the labels for the MV plane, PV orifices, and LAA orifice. LA volumes and surface areas were computed from the mesh objects.

### II.E. The LA Long Axis

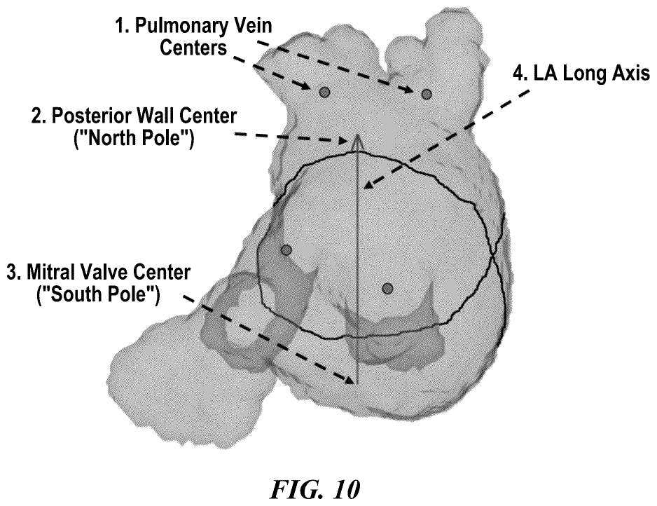

To begin the LAPS system framework, a central LA long axis (L) is first defined as a vector between the centroid of the mitral valve plane and center of the posterior wall (FIGS. 5A, 5B, and 10). The posterior wall center is chosen as the point on the mesh closest to the Euclidian centroid of all PV plane centers. If a subject has a right or left common PV, then the centroid of the common PV is weighted double in the calculation to keep the posterior wall point centered. This helps the process remain independent of the number of PVs.

FIGS. 5A and 5B show a LAPS coordinate system framework, according to embodiments of the present technology. FIG. 5A shows a sample LA body surface mesh with MV plane and PV orifices indicated by shadows. The solid dots denote the mitral valve (MV) centroid, pulmonary vein (PV) centroids, and posterior wall center, as labeled. The 3 orthogonal vectors L, R, and P computed from these landmarks define the prime meridian. FIG. 5B shows lines of longitude and latitude plotted at 10% increments on the LA endocardial surface. This demonstrates how normalized distance coordinates conform to the shape of the atrium.

FIG. 10 shows a LA long axis defined using an arbitrary LA geometry, shown in a left-oblique view with a transparent surface.

### II.F. The Prime Meridian

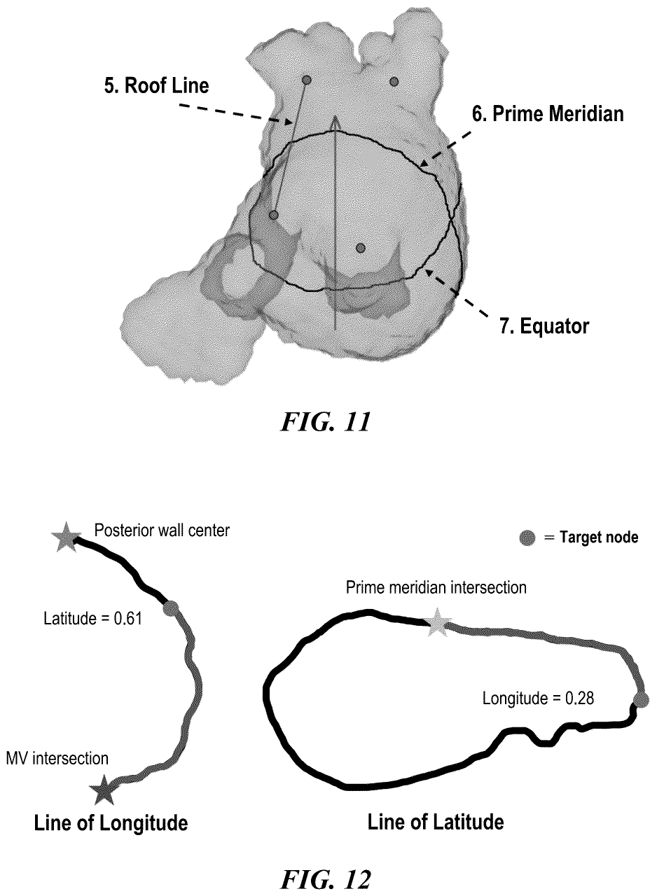

A roof line vector (R) is defined by connecting the centroids of the right to the left superior PVs (FIGS. 5A, 5B, and 11); if the patient has a single PV on either the right or left, the centroid for the single PV is used. The cross product of L with R produces a direction reference vector (P), which points perpendicularly from the LA long axis towards the inferior wall. The mesh is intersected by the plane formed by L and P. The prime meridian contour is the mesh-plane intersection in the direction of P. This is the first line of longitude, and it bisects the LA into the left and right halves. FIG. 11 shows a roof line, prime meridian, and an example line of latitude (the “equator”), according to embodiments of the present technology.

It is understood that the determination of the prime meridian as described above is for illustration purposes only and not intended to be limiting. Another line of longitude determined as described below may be designated as the prime meridian.

### II.G. Lines of Longitude

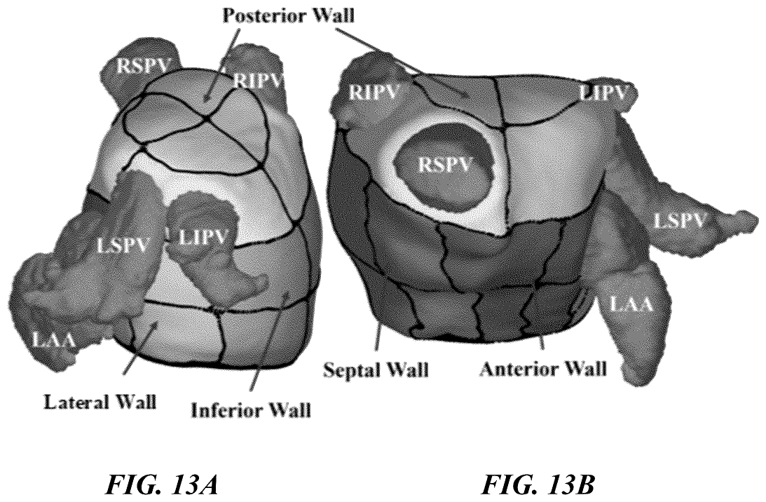

A line of longitude is calculated for each node in the surface mesh: first, a plane is defined using the target node, the posterior center, and the mitral valve center. The geodesic path, as a line of longitude through the target node, from the posterior center to the mitral valve plane through the target node is calculated as the intersection of the plane with the mesh. The latitudinal coordinate (latitude changes along a line of longitude) for the target node is the ratio of the arclength to the target from the mitral valve by the entire arclength between the mitral valve and posterior center. This normalizes the coordinate values from 0 to 1 (FIGS. 8 and 12).

FIG. 8 shows LAPS coordinates displayed on a gallery of LA geometries, according to embodiments of the present technology. Latitude and longitude coordinates are each normalized from 0 to 1, yielding a consistent 2-dimensional description of LA surface location across subjects. There is a longitude discontinuity at the prime meridian. The jagged appearance is an artifact of plotting nodal values on a discrete mesh; the underlying coordinates meet exactly at the prime meridian. LA morphology varies significantly: (from left) standard configuration, inferiorly located LAA, standard configuration with extreme dilation, common left PV, missing LAA.

FIG. 12 illustrates determination of LAPS coordinates for a single target node, according to embodiments of the present technology. The arclength from the mitral valve intersection to the target node is divided by the total length of the path from the mitral valve intersection to the posterior wall center to achieve a normalized coordinate value.

### II.H. Lines of Latitude

After latitude values (or latitude coordinates) have been calculated on all the meridians, lines of latitude are computed as their isolines of equal latitude. The parallels (lines of latitude) are constructed in the orthogonal direction. For a target node, a parallel contour line is drawn connecting all points on the edges of the mesh which share that target node's latitude value. For example, if the target node has a latitude of 0.563, there most likely is no other node with exactly that value. However, there may be an edge with nodes which have latitudes of 0.555 and 0.571. The exact Euclidean point for the line of latitude is determined by interpolating those values along the edge to find the target latitude. This contour intersects the prime meridian, and the intersection marks the reference point for calculating longitude coordinate values on the parallel. The longitude value is the distance from the prime meridian to the target node, divided by the circumference of the contour. At the prime meridian, the longitude is discontinuous from 1 to 0 (FIG. 8).

Once the line of latitude is drawn, the longitudinal coordinate is calculated along it by dividing the arclength to the target node by the circumference of the contour (FIG. 12). The arclength starts where the prime meridian intersects the contour. At that location, the longitude is both 0 and 1.

### II.I. Coordinate Computation

The longitude value (or longitude coordinate) of a target node (or point) relates to a distance between the line of longitude through the target node and a prime meridian. For instance, the longitude value of a target node (or point) relates to a normalized distance between the line of longitude through the target node and the prime meridian. The longitude values of nodes on a same line of longitude may be the same.

The latitude value (or latitude coordinate) of a target node (or point) relates to a distance between the target node and the first or second reference point along the line of longitude through the target node. For instance, the latitude value of a target node (or point) relates to a normalized distance between the target node and the first or second reference point along the line of longitude through the target node. The latitude values of nodes on a same line of latitude may be the same.

LAPS coordinates (FIG. 8) were computed as described for every node on the mesh generated at the LA reservoir phase for each of the 45 4DCT scans. The reservoir phase was identified as the frame having the largest LA volume.

### II.J. Automatic Region Assignment

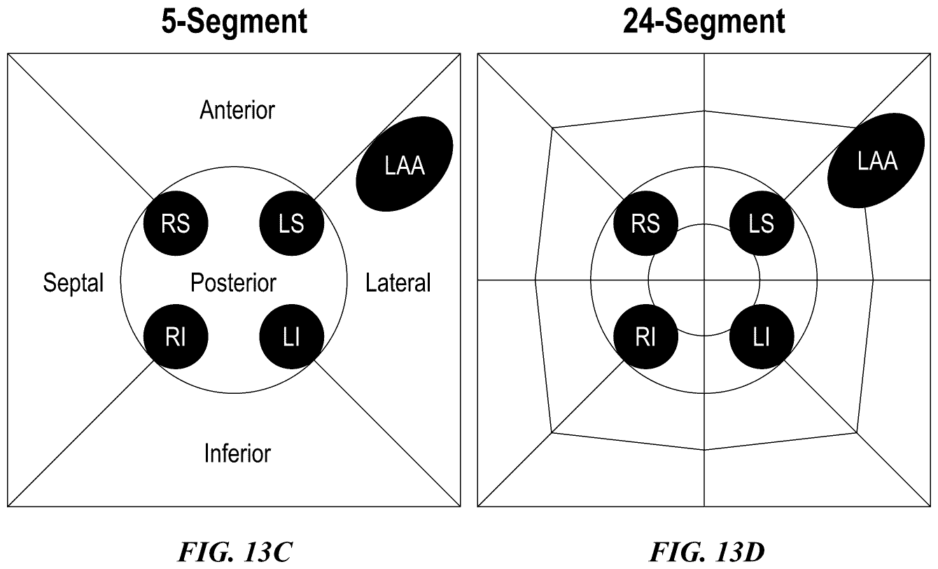

The LA is commonly divided into 5 walls: the anterior, posterior, septal, lateral, and inferior walls. FIGS. 13A-13D show LAPS coordinates used to automatically divide and display the LA body into subregions, according to embodiments of the present technology. FIGS. 13A and 13B show the LA surface may be partitioned into 5 walls. Each side wall is further subdivided into 4 segments, and the posterior wall into 8 segments. FIGS. 13C and 13D show LA bullseye plot layout for the 5-wall and 24-segment models, respectively. The locations of the PVs and LAA on the plot are representations of the territories typically occupied by each feature. However, the LAA is always on the lateral wall and the PVs on the posterior wall.

LAPS coordinates may be used to automatically divide an LA mesh into relevant anatomic subregions. First, the node of each PV orifice with the smallest latitude is identified, which is the point on the PV closest to the mitral valve. If an LAA orifice plane is available, then the LAA-labeled node with the largest longitude is identified, which is the most anterior point of the LAA. Linear boundary equations are defined in LAPS coordinates that connect the identified PV points to each other. An additional line segment is computed to connect the left superior PV (LSPV) to the LAA. These boundaries are used with a series of conditional statements to classify all nodes into the appropriate wall subregion. For example, the septal wall is defined as nodes with longitudes between the right superior PV (RSPV) and right inferior PV (RIPV) and latitudes less than the RIPV-RSPV boundary. Each region may be further subdivided to user specifications or until reaching the limit of mesh resolution (FIGS. 13A-13D).

### II.K. 2D Visualization

In keeping with the analogy to global cartography, the Earth's surface has traditionally been projected into planar 2D maps. Although LAPS coordinates are not mathematically equivalent to latitude and longitude, they may still take advantage of cartographic projection algorithms to enable flexible 2D data exploration. LAPS coordinates were visually evaluated with 71 packaged cartographic projection algorithms in the MATLAB Mapping Toolbox, as well as an implementation of the Peirce-Quincuncial (PQ) projection. Triangular surface textures were applied to the LA surface and projected using the same algorithms so that the local effects of shape and size distortions may be visualized.

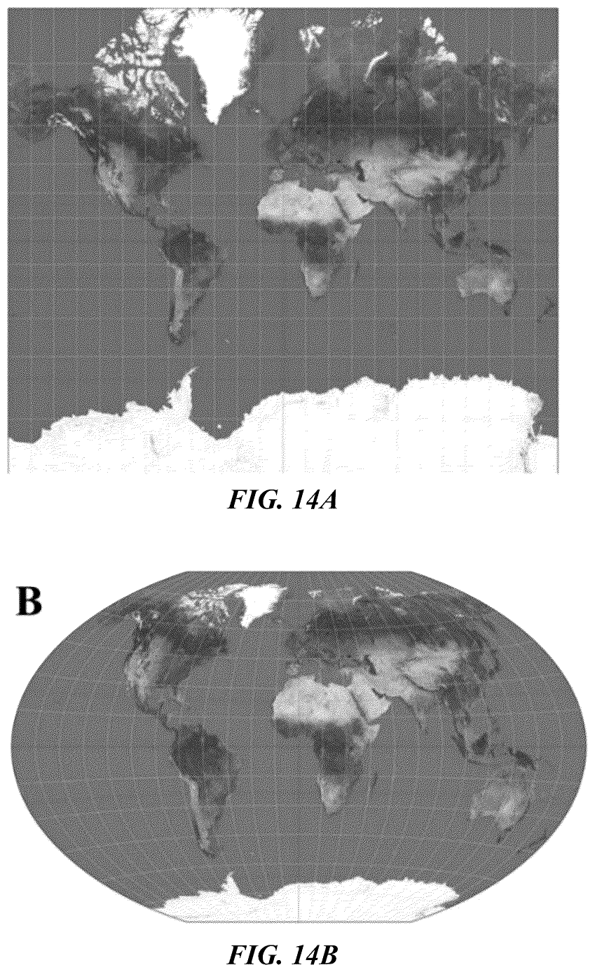

In brief, it is infeasible to perfectly represent a spherical object on a 2D surface. All maps need either distort the distance, area, or shape of features; many distort all 3 as a compromise. Each projection has its own use. For example, the most famous map projection is the Mercator, which preserves the angles between meridian lines at the cost of large size distortions. This made the Mercator map excellent for nautical navigation but instigated political drama, as some countries appear misleadingly small compared to others. Many maps invented during the European voyages across the Atlantic Ocean place Europe in the center of the map, but this is mathematically an arbitrary choice. FIGS. 14A-14D illustrates a gallery of exemplary world map projections: FIG. 14A Mercator, FIG. 14B Winkel-Tripel, FIG. 14C Goode Homolosine, and FIG. 14D Azimuthal Equidistant.

Because the LA has not been approximated as a sphere, the projections may not make mathematical sense, and the degree of distance, shape, and area distortion will depend on how far an individual atrium deviates from a sphere. However, using cartographic projections, LA data can be rapidly visualized in many forms, whereas prior LA coordinate systems have only one demonstrated projection. The MATLAB Mapping Toolbox has 71 available map projections.

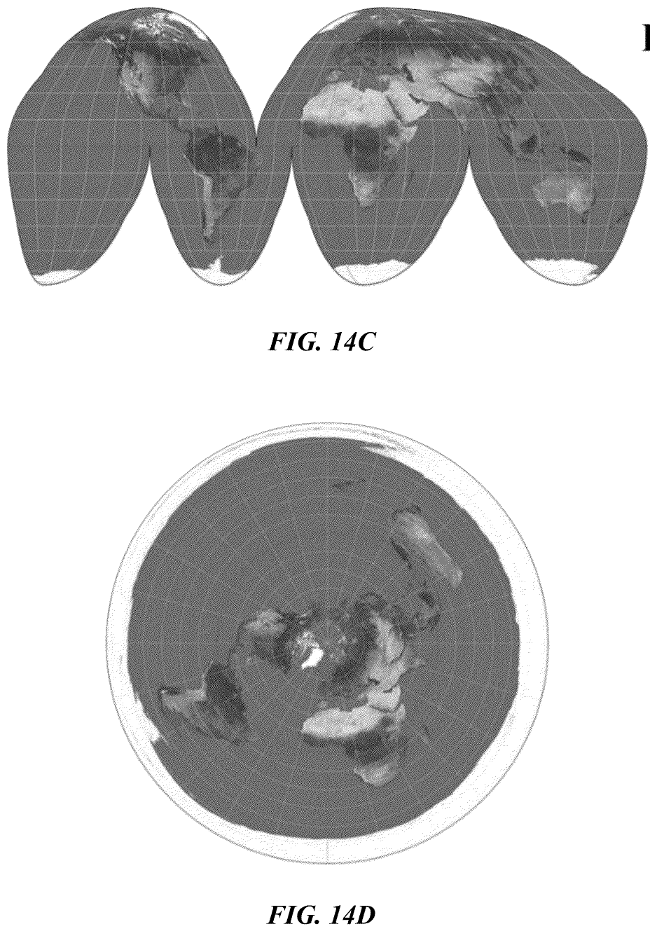

FIG. 15 demonstrates a gallery of scalar strain data from a left atrium, plotted using various cartographic projections, according to embodiments of the present technology. LSPV=left superior pulmonary vein. LIPV=left inferior pulmonary vein. RSPV=right superior pulmonary vein. RIPV=right inferior pulmonary vein. LAA=left atrial appendage. MV=mitral valve.

All LA subregions can be displayed on a standardized square diagram (FIGS. 13A-13D). The diagram follows the spatial arrangement of a PQ projection of a sphere. The mitral valve is represented by the outer edges, and the edges of the posterior wall by an interior circle. Each of the other 4 LA walls are represented in a quadrant of the square. This is the “bullseye plot” for the left atrium, used to summarize data on the LA surface in a 2D plot.

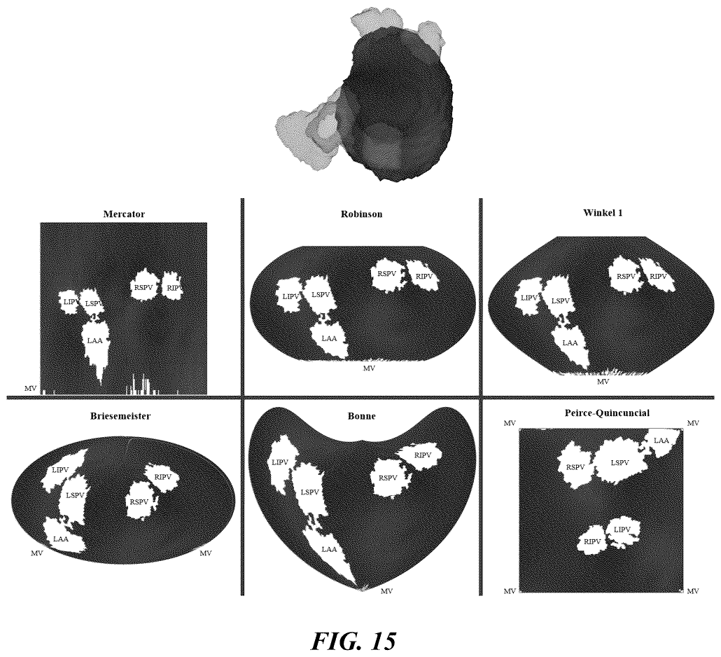

Of the many possible projections, the Peirce-Quincuncial has stood apart as being particularly useful for viewing LA strain data. The algorithm unfolds the shape into a square, splitting and diminishing the mitral valve to the corners. The four sides of the square naturally align with the LA's typical four pulmonary veins and the associated four walls: anterior, septal, lateral, and inferior (sometimes called the “floor”). The centered posterior wall and PVs are convenient for electrophysiology applications, since those are the most common targets for catheter ablation of arrhythmogenic substrate. The Peirce-Quincuncial projection is used as an exemplary data presentation tool. FIG. 16 demonstrates the Peirce-Quincuncial projection algorithm, implemented in MATLAB.

### II.L. One-Way Point Transfer Validation

The accuracy of the coordinate system was evaluated by computing the one-way Euclidian distance error of transferring points from a Mesh A to a second Mesh B, similar to the method described in the validation of the biventricular coordinate system, Cobiveco. However, rather than creating a statistical shape model from the coordinates of A and B to serve as a ground truth, a model with known coordinate values was formed by performing non-rigid registration from A to B. This ensures that the shape differences between A and the ground truth represent the full range of differences between two real image datasets, whereas a statistical shape model results in a ground truth shape that is an average of A and B.

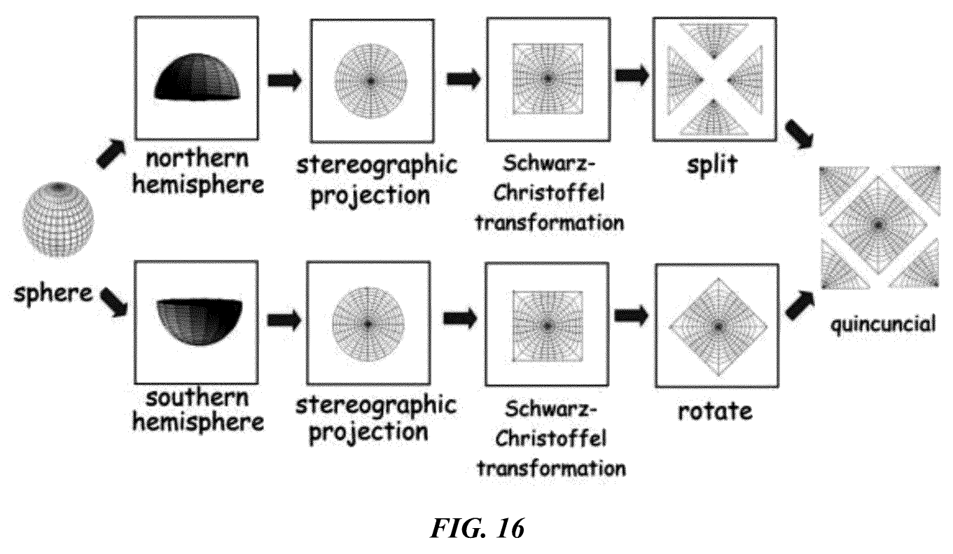

FIG. 17 shows a one-way error validation process, according to embodiments of the present technology. Ground truth LAPS coordinates are obtained on Mesh C by registering A to B with coherent point drift (CPD), and test LAPS coordinates are calculated on Mesh C in isolation. Ground truth LAPS coordinates are mapped into the same LAPS space as the test coordinates using their point correspondence. Converting back to Cartesian by barycentric interpolation on the faces of Mesh C yields a Euclidian distance separation between truth and test points. More specifically, the validation may proceed as follows:

Compute LAPS coordinates for the nodes of Mesh A.

Gross alignment: Perform affine registration from Mesh A to Mesh B with coherent point drift (CPD) so that their pulmonary veins orifices are aligned, yielding Mesh A′. This aligns the meshes to a datum so that A′ and B have the same orientation.

Proceed with non-rigid registration from A′ to B using CPD yielding Mesh C which has direct point correspondence to A.

Copy the original LAPS coordinates from the nodes of A to corresponding nodes of C. These are the ground truth coordinates.

Compute new LAPS coordinates on C. These are the test coordinates.

Map the ground truth LAPS coordinates into the same LAPS space as the test coordinates using their point correspondence.

Mapping LAPS back to Cartesian: For each node of C, convert truth LAPS coordinates into Cartesian coordinates on the surface of C. Use barycentric interpolation on the faces of the mesh.

Calculate the Euclidian distance errors between the ground truth points and their corresponding test points.

One-way mapping validation was conducted in Groups 1 and 2 (see Tables 1 and 2). First, in each subject of Group 1 all nodes of the mesh were transferred from the scan taken before the ablation procedure to the scan taken after, and vice versa. The median and mean Euclidian distance error in millimeters was recorded for each mapping. This represents the intrasubject error. Likewise in Group 2, one-way error was computed between each pair of subjects in cohorts A and B, in both directions, capturing the intersubject error.

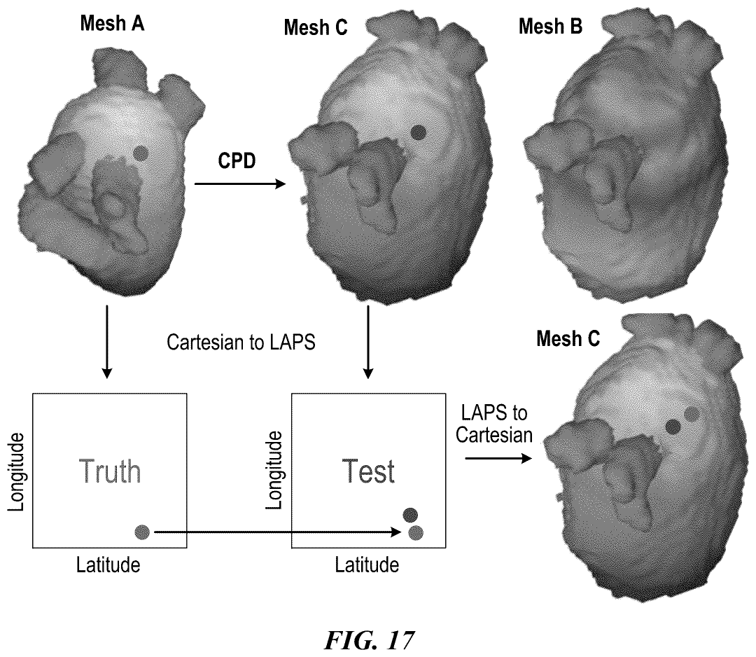

Two 3D left atrium meshes are registered to each other using a probabilistic non-rigid registration method. This creates trackable point correspondence between shapes. Once Mesh A is registered to Mesh B, creating a new Mesh C, coordinates are calculated for the nodes in Mesh A and Mesh C. Because the nodes have direct correspondence, the error in 2D coordinates may be computed (FIG. 18).

In Group 1, transferring all nodes from the pre- to post-ablation geometries resulted in an average median error of 2.13 mm (Table 2). Mapping points from the second to first scans was significantly more accurate than mapping in the forward direction (p<0.01). In Group 2, the average median error between all pairs was 3.99 mm, despite the variety of sizes and geometries paired together (Table 2). One-way transfer error was not correlated with the degree of remodeling nor the LA volume or surface area.

TABLE 2
ONE-WAY ERROR VALIDATION RESULTS

| | # of | Median Error | Mean Error |
|---|---|---|---|
| Test Group | Comparisons | (mm) | (mm) |
| Group 1 | 15 | 2.67 | 3.47 |
| Pre- to Post-Ablation | | | |
| Group 1 | 15 | 1.59 | 2.08 |
| Post- to Pre-Ablation | | | |
| Group 1, All | 30 | 2.13 | 2.77 |
| Group 2 | 15 | 3.96 | 4.38 |
| Cohort A to B | | | |
| Group 2 | 15 | 4.02 | 4.48 |
| Cohort B to A | | | |
| Group 2, All | 30 | 3.99 | 4.43 |

Results reflect the one-way transfer error in Euclidian distance for all nodes in the meshes. The reported median and median are averaged across all comparisons in each test group.

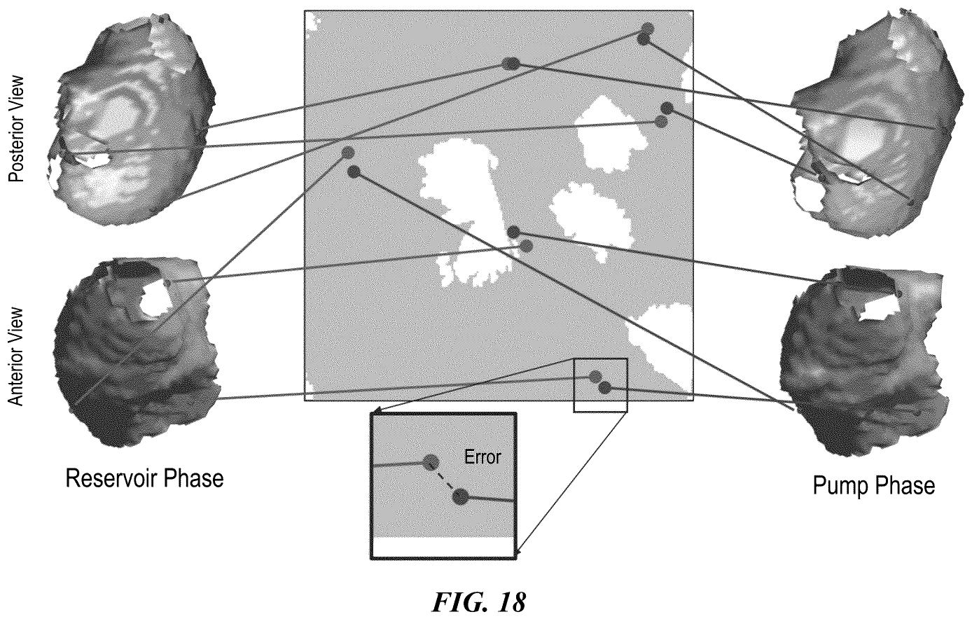

FIG. 19 shows one-way transfer errors for Group 1 averaged across scans and projected onto a Peirce-Quincuncial square. The posterior wall tended to have smaller median errors but larger variability than other segments. Large longitude errors near the prime meridian are an artifact of the coordinate discontinuity; they do not translate into significant Euclidian distance error.

### II.M. Sensitivity Analysis

A sensitivity analysis was conducted to determine the impact of variation in plane cuts on landmark definition and coordinate assignments. In 3 sample subjects and for 100 iterations per subject, each of the PV centroids were randomly translated up to 5 mm in a random direction. The MV plane was randomly tilted up to 15 degrees in a random direction. One-way point transfer was used to evaluate the deviation in LAPS coordinates of each iteration compared to the original neural-network-based landmarks.

As a result of randomly perturbing the PV and MV landmarks, the posterior wall center shifted a median of 1.4 mm (range: [0.6 mm, 6.2 mm]) from its original location. The distribution was heavily skewed toward 0 because the definition from the spatial average of the PVs keeps the posterior wall landmark stable. The LA long axis L deviated by a median of 3.4 degrees (range: [0.5 deg, 13 deg]). The direction reference vector P deviated by a median of 2.2 degrees (range: [0 deg, 17 deg]). Median one-way transfer error was 2.9 mm, with a mean of 3.9 mm. LAPS coordinates were found to be reasonably insensitive to variation in plane cuts.

The coordinate transfer error was relatively small, and the anatomic structures map coherently to the 2D coordinate space, so this provided the impetus to proceed with further LAPS applications.

### II.N. Coordinate System Applications

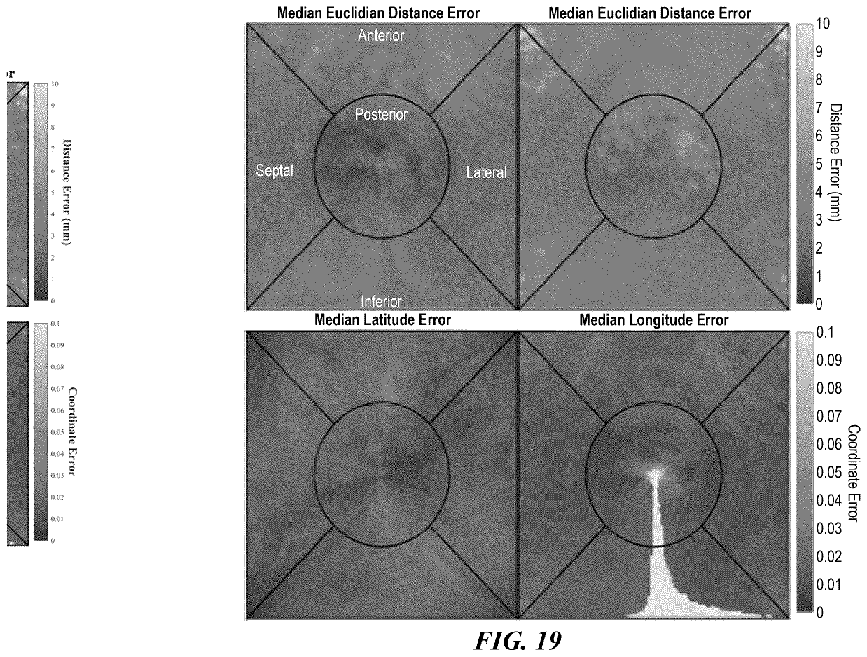

The left atrium may be divided into subregions related to its anatomy, like the LV for its bullseye plots. The names and definitions for major LA regions are not exactly standardized, but the LA is most commonly divided into 5 walls, which are the anterior, posterior, septal, lateral, and inferior walls. FIG. 20 shows a visualization of LA wall labels with respect to relevant anatomy. The anterior wall is the segment bounded by the superior PVs and the mitral valve. The septal wall is bounded by the right PVs and mitral valve. The inferior wall is bounded by the inferior PVs and mitral valve. The lateral wall is bounded by the left PVs and mitral valve. The lateral wall boundary goes around the LAA anteriorly to make the size of the wall more equal to the others, and to consistently place the LAA in the lateral wall segment. The posterior wall is bounded by the outer perimeters of all the PVs.

The LAPS implementation in MATLAB uses the computed LAPS coordinates for an LA mesh to automatically divide the mesh into relevant anatomic subregions. First, the node of each PV plane with the smallest latitude value is identified. These are the closest points of the PVs to the mitral valve. If an LAA plane is available, then the LAA node with the largest longitudinal value is identified. This is the most anterior point of the LAA. Two-dimensional linear equations are defined connecting the PVs to each other and to the mitral valve using their LAPS coordinates. An additional equation is used to connect the LSPV to the LAA. These equations are used with a simple series of conditional statements to label all nodes with the appropriate subregion. For example, the septal wall is defined as nodes with longitudes between the longitudes of the RSPV and RIPV and latitudes less than the RIPV-RSPV equation. Although the boundaries are linearly defined, because the coordinates were defined on a 3D shape, the resulting region boundaries are curved to match the anatomy of the LA (FIG. 20).

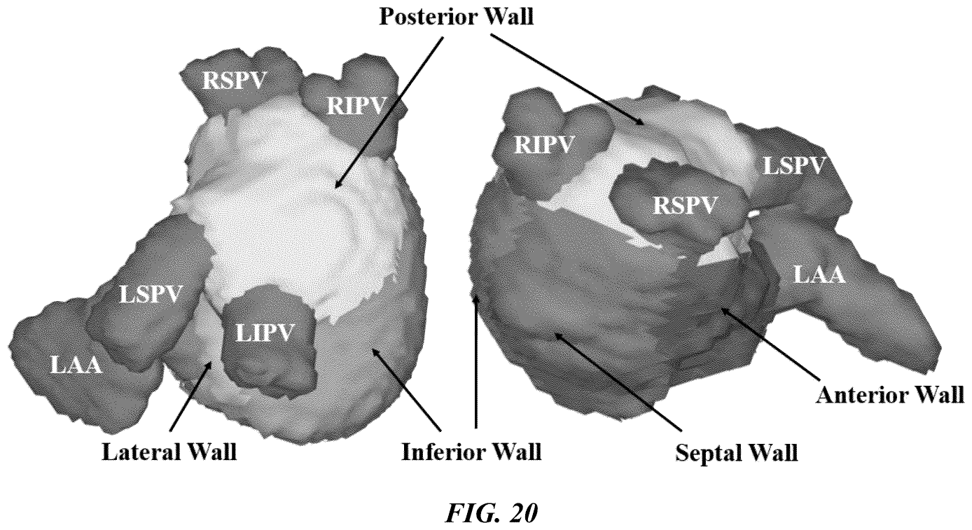

Furthermore, each region may be further subdivided, infinitely or until reaching the limit of mesh resolution. This may be done by again utilizing the linear boundary equations to equally divide a region to a user-specified resolution. For example, the anterior wall has a regional segmentation label of “1.0”, and the user wishes to divide this into 4 equal segments. In this case, the LSPV-LAA-Mitral equation is subtracted from the RSPV-Mitral equation to obtain the range of latitude and longitude coordinates spanning the anterior wall. The range is divided into 2 halves with equal latitude range and 2 halves with equal longitude range, totaling 4 segments. Each segment is in turn assigned a unique numerical label, like “1.1, 1.2, 1.3, 1.4.” FIG. 21 shows subdivisions of the LA walls, where this 24-segment model divides each of the side walls into 4 subregions and the posterior wall into 8 subregions.

Subdividing the posterior wall is unique, because all meridians converge on the posterior center. A division into 4 subregions results in an outer ring with 2 segments and an inner circle with 2 halves. To keep segments associated with the 4 pulmonary veins, the posterior wall is split into 8 segments—an outer ring with a segment for each PV and an inner circle with a quadrant for each PV.

The subdivision process is generalizable to enable infinite divisions of the LA walls into perfect square numbers of segments. Non-square divisions are possible but not available in the current MATLAB implementation. Table 3 shows the possible number of segments available for automatic subregion assignment. The subdivision process fails when any of the attempted subregions becomes smaller than an element of the mesh. Through experience processing hundreds of left atria through LAPS, this almost always happens first at the lateral wall, which is the smallest wall, especially if the LAA is missing. Over 99% of scans processed with a mesh element size of 2 mm can handle 24-segment models, and most can also produce 54 segments.

TABLE 3
Subregion assignment and calculation for
determining number of LA segments.

| Perfect Square | Side Wall | 4 × Side | Posterior Wall | Total |
|---|---|---|---|---|
| Index | Subdivisions | Wall | Subdivisions | Segments |
| 0 | 0 | 0 | 0 | 1 |
| 1 | 4 | 16 | 8 | 24 |
| 2 | 9 | 36 | 18 | 54 |
| 3 | 16 | 64 | 32 | 96 |
| 4 | 25 | 100 | 50 | 150 |
| 5 | 36 | 144 | 72 | 216 |

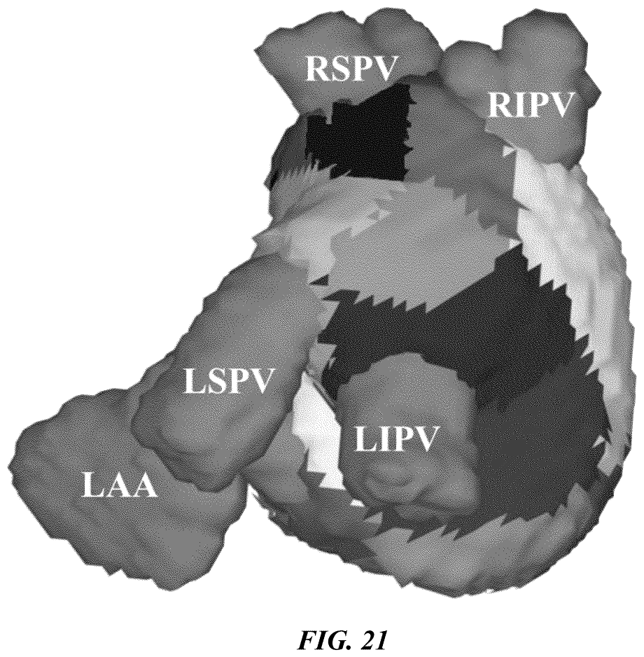

FIGS. 22A and 22B illustrate LA Bullseye plots for 54 and 96 segments, respectively.

### II.O. The LA Bullseye Plot

Once each node is assigned a subregion, scalar data can be summed or averaged over the nodes in a region. The single resulting value is assigned to the segment, and all segments can be displayed on a standardized Peirce Quincuncial square. This is the “bullseye plot” for the left atrium, used to summarize data on the entire LA surface in a 2D plot.

With the introduction of the LA bullseye plot comes much of the same functionality as for the LV. Scalar data for any individual patient may be displayed in a standardized way for medical charting and diagnostics. Relevant anatomic zones may be marked and targeted for therapies. For example, the segments of the inner circle are linked to the pulmonary veins. One might imagine LGE-MRI scar tissue, electroanatomic mapping voltages, and local strain magnitudes may be displayed, overlaid, and merged on the LA bullseye.

Of increasing importance in the age of big data is the creation of powerful statistical atlases to differentiate important patient groups. To date, this has been a near impossibility for data collected in the left atrium due to the lack of a fast, fully automated common coordinate system. With this development, if a cohort of patients are segmented into subregions using LAPS, it is computationally trivial to calculate group means and statistical distributions in each segment.

To create the LA bullseye plot (FIGS. 13A-13D), segments were assigned before the 2D projection to avoid the projection's area distortions. On average, when the atria were subdivided into 24 segments, the smallest width or height of any segment was approximately 10 mm (std=2 mm). The smallest segment was most commonly in the lateral wall, especially in the case of a missing LAA. The smallest segment dimension of 10 mm for a 24-segment model is a factor of 2.5 larger than the interpatient one-way error, which may justify the reasonable use of a 24-segment division of the LA surface to display aggregate data in each region. Further subdivisions are possible. For example, FIGS. 22A and 22B illustrate LA Bullseye plots for 54 and 96 segments, respectively. However, signal to noise limitations in estimating physiological parameters may occur if the size of the segments becomes too small.

### II.P. Additional Applications: Derivatives, Data Sampling, Shape Modeling, and 2D Visualization

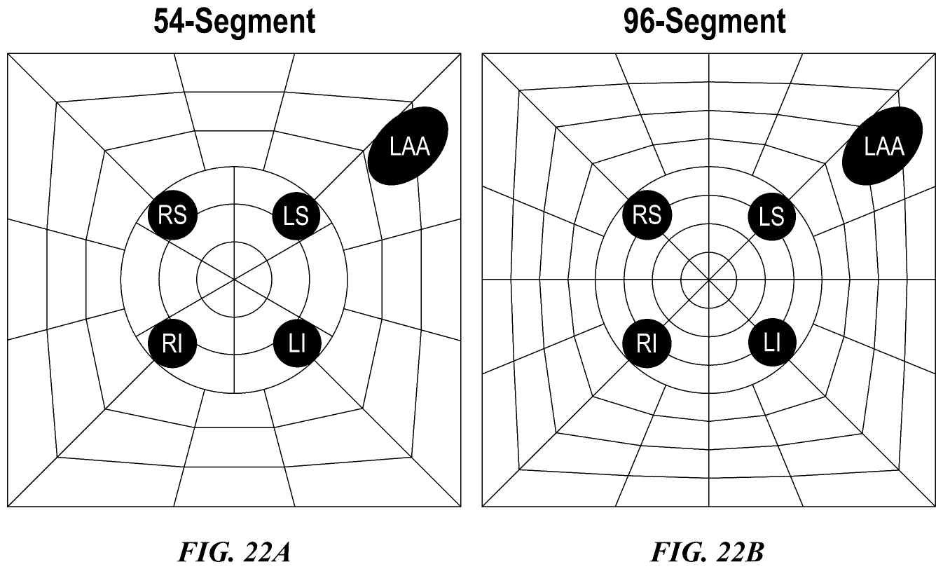

In an orthogonal coordinate system, isolines of each coordinate intersect each other at right angles at every point on the shape. Geodetic coordinates as applied to spheres have this property, and by design LAPS should as well. The longitudinal coordinate was defined as the isolines of the latitude field. If the latitude field is a true gradient field, then the system is constrained to be orthogonal since gradient vectors are orthogonal to their isolines. Definitive claims that the latitude field is a gradient field might be avoided, since the coordinate was not defined with a continuous function. However, after visual inspection of the continuity of the latitude across the shape (FIG. 23), it is clearly a close approximation. FIG. 23 shows LAPS coordinates determined according to embodiments of the present technology. An orthogonal system allows for simple calculation of spatial derivatives across the surface, which may have exciting implications in electrophysiology, such as identifying electrical rotors as targets for ablation.

Another advantage of a highly accurate coordinate system is the ability to sample arbitrary shapes. A single coordinate point may serve as an anatomic landmark, common amongst all left atria. Without calculating LAPS coordinates for an entire shape, meshes can be sampled in particular areas of interest, or evenly sampled to characterize the global shape of the LA. Much interest has been taken in statistical shape modeling (SSM) of the LA, and some modes of shape variation have been shown to indicate ablation success. Particle-based modeling involves computationally expensive optimization of point distribution across a population of meshes, all to maintain the point correspondence that is inherent to a coordinate system. LAPS may feasibly be used to speed the computation of large SSMs of the LA. The same principle may be applied to registration algorithms—current registration algorithms optimize registration by learned mutual information, but the provision of a few common coordinate points drastically reduces the complexity of registration tasks.

Mesh nodes assigned LAPS coordinates were transformed to 2D space using 72 cartographic projection algorithms. The PQ projection was chosen as the basis for presenting LA surface data; the four sides of the PQ square naturally align with the LA's typical four pulmonary veins and the associated four walls: anterior, septal, lateral, and inferior. It conveniently maps the pulmonary vein locations toward the center while placing the mitral valve at the corners. The PQ projection for a globe was designed to be a tessellating, conformal map. The distortion properties of map projections are commonly assessed by creating regular texture patterns on a sphere and observing how the projection changes the pattern's shape, size, and density.

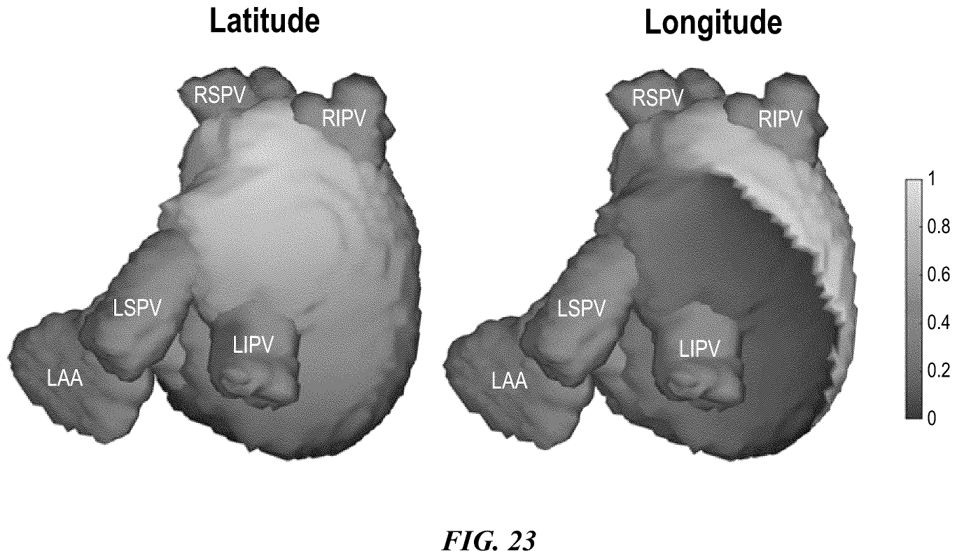

FIGS. 24A-24C show Peirce-Quincuncial projection warps the LA surface similar to its intended use in global cartography, according to embodiments of the present technology. FIG. 24A shows a left atrial body covered in a regular triangular surface texture created from the mesh faces. FIG. 24B shows textured LA body surface mapped to a square using LAPS coordinates and the Peirce-Quincuncial projection. FIG. 24C shows Peirce-Quincuncial projection of a world map covered in a uniform circle texture. Note the similarities in the warping patterns in FIGS. 24B and 24C.

### II.Q Exemplary Implementation

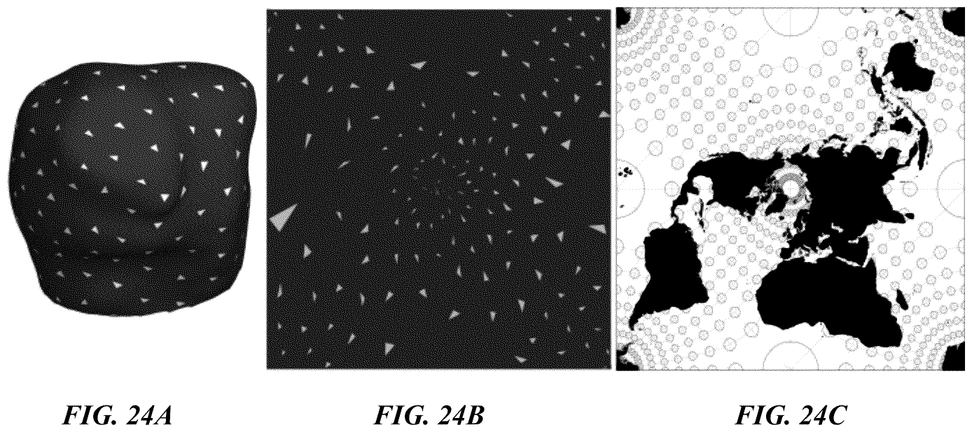

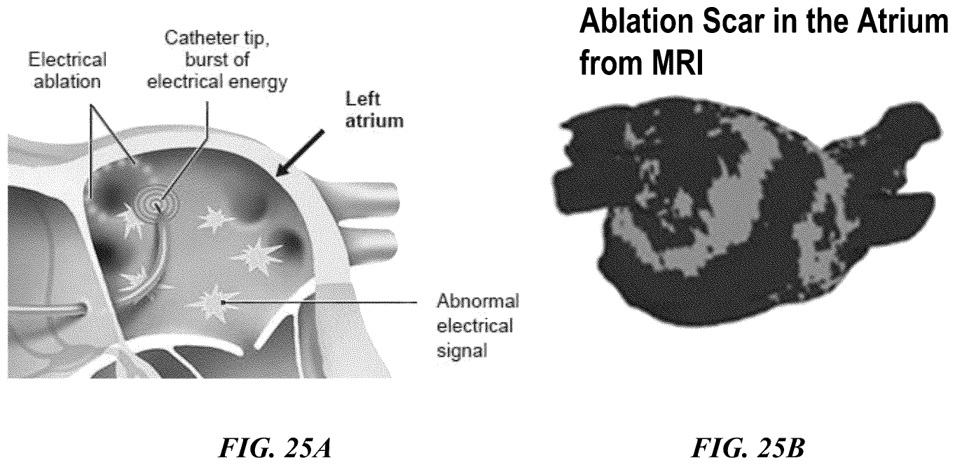

Atrial fibrillation (AF) is the most common cardiac arrhythmia, affecting 60 million people globally. Catheter ablation is a common treatment for AF, creating electrically inert, stiff scar tissue to correct the arrhythmia. FIG. 25A illustrates a schematic representation of AF. However, it has a high failure rate (˜40%). Magnetic resonance imaging (MRI) is state-of-the-art to measure scar tissue to plan the procedure and assess the outcome. FIG. 25B depicts a schematic view of an ablation-induced scar in the left atrium, as identified using magnetic resonance imaging (MRI). MRI scans are costly and lengthy, so these scar assessments are not often performed. In contrast, 4-dimensional computed tomography (4DCT) is cheaper and much faster than MRI.

The technology disclosed herein provides a method to compare LA surface strain from 4DCT in the regions of ablation scar, from images collected before an after the ablation procedure. Retrospective study of 15 patients with 4DCT before and after ablation. Ablation locations were recorded by hospital staff. Seven of the 15 patients were imaged while in AF—treated as a separate category.

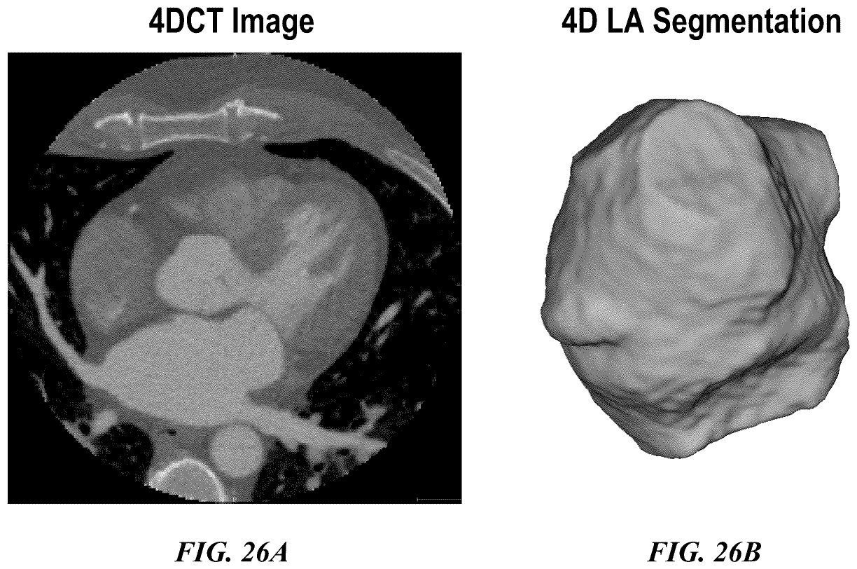

FIG. 26A shows an exemplary 4DCT image of a patient. FIG. 26B shows a result of 4D LA segmentation using a trained CNN model. FIG. 26C shows a 4D triangular mesh generated based on the result of the LA segmentation as in FIG. 26B. FIG. 26D illustrates mesh triangles warp over time. Regional Shortening (Strain) Calculation derived from 4DCT imaging data (RSct) can be employed to quantify how much a region of the left atrium (LA) contracts or shortens over time. RSct at vertex v and time t may be calculated according to Equation (1), where A=area of the triangular face of a triangle node of the surface mesh.

$$R {S}_{CT} ( v , t ) = \sqrt{\frac{A ( v , t )}{A ( v , 0 )}} - 1. \qquad ( 1 )$$

FIG. 18 shows a 3D LA shape projected onto a Peirce Quincuncial square, according to embodiments of the present technology. The mitral valve is pushed to the corners, with the pulmonary veins centered and the LA appendage antero-lateral. Point transfer from reservoir (max LA size) to pump phase (min LA size) had a minimal error.

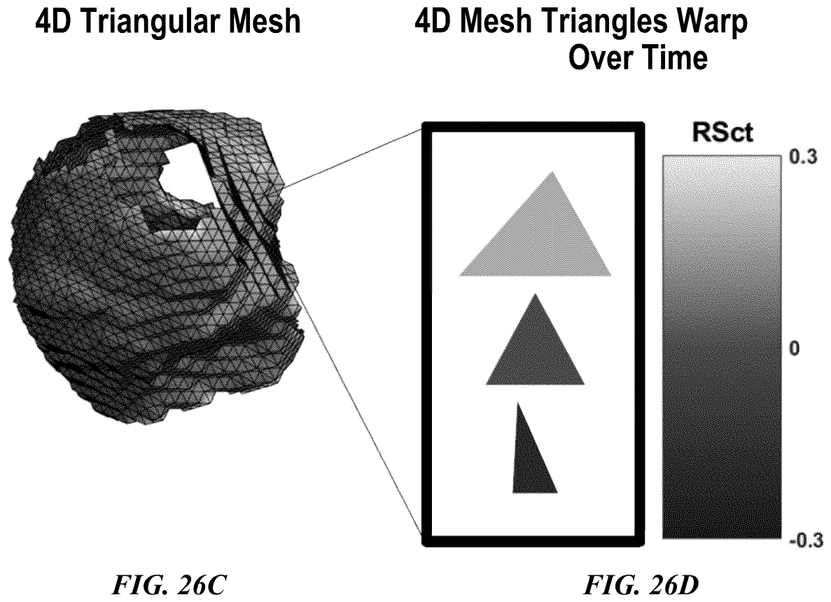

FIG. 27 shows the change in RSct strain in ablated versus non-ablated regions. Asterisks (*) denote statistically significant differences (p<0.05). When considering all 15 subjects, paired t-test results found no particular wall to have any significant reduction in RSdr after ablation. However, when considering the 8 subjects who were in sinus rhythm during both scans, the relative regional strain change after ablation was −16.3% in the septal wall (p=0.030) and −18.3% in the posterior wall (p=0.027). No statistically significant reductions were observed in the anterior wall (−10.1%, p=0.062), inferior wall (−11.3%, p=0.051), or lateral wall (5.6%, p=0.299).

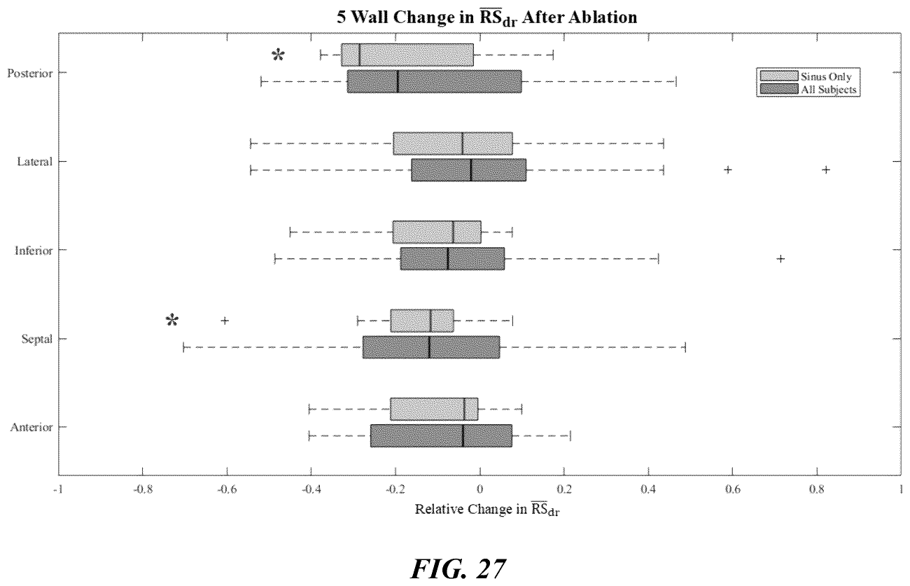

FIG. 28 illustrates the functional consequences of ablation on regional strain using RSCT maps derived from the 4DCT data as discussed in FIGS. 26A-26D. Row 1 shows a 2D representation of the left atrium (LA), with the colored segments delineating the ablation patterns based on the intra-operative notes (red=ablated, blue=not ablated, white=unclear, red ‘X’=LAA exclusion). Rows 2 and 3 present the RSCT maps for four subjects, before and after ablation, respectively. Ablated regions saw an average 19.8% relative reduction in RSdr, while non-ablated regions decreased only 3.5%. The effect of ablation was significant, causing an estimated 15.3% relative reduction in RSdr accounting for random effects (p=0.012, Cl: [−27.2%, −3.4%]). Post-ablation strain maps reveal distinct regions of low strain that align with the reported ablation patterns, particularly forming characteristic rings of reduced strain around the pulmonary veins. These spatial patterns confirm the expected physiological impact of ablation as reflected in the RSCT metric.

### III. DISCUSSIONS

The coordinate system for the left atrium disclosed here—the Left Atrial Positioning System is designed to be fully automated and improve or maximize accuracy of correspondence between arbitrary LA shapes. It provides a variety of data display options, including a LA bullseye plot for standardized visualization.

### III.A Accuracy Validation

One-way transfer error is an appropriate test of a coordinate system's accuracy because in practice imaging data is mapped from one study to another. Cobiveco used one-way error for validation, with a resulting accuracy of 1.17 mm median distance error between different patients in the left ventricle. The Universal Ventricular Coordinate (UVC) system was also tested, measuring 5.93 mm error. In comparison to these LV systems, LAPS is reasonably accurate with a median intersubject one-way error of 3.99 mm on validation atria which were significantly larger than a healthy LA (Table 1). The accuracy was insensitive to the magnitude of LA shape differences (FIG. 9), which is a key benefit of using anatomy-specific coordinate systems over general registration algorithms.

### III.B. Follow-Up LA Imaging Studies

LAPS presents a potential improvement to data integration in multimodality and follow-up LA imaging studies where the LA has experienced significant remodeling. Currently, multimodality images are combined either as a side-by-side comparison on a medical monitor or with image registration. The latest version of Carto 3 mapping system (Biosense Webster) utilizes a landmark-based registration algorithm to align LA surface meshes. Such registration algorithms are subject to non-negligible errors from variation in selecting landmark points, atrial alignments, and cardiac gating errors. Landmark affine, iterative closest point, and other non-rigid algorithms have been measured to have 1.8 to 4.6 mm pointwise distance error in the left atrium. In comparison, the one-way intrasubject LAPS transfer error was 2.13 mm (Table 2), even though the follow-up atria were significantly remodeled (FIG. 9). LAPS may be used to register LA surface data by computing coordinates on both a template and target mesh and assigning template data values to the target points with matching coordinates.

### III.C. Cohort LA Imaging Studies

Whereas intrapatient image comparisons are commonplace, the ability to compare regional LA surface data between different patients is an outstanding challenge in cohort studies. Aligning interpatient LA geometries with non-rigid image registration can show variable performance dependent on the template mesh used or be time-consuming with exacting definition of landmark points and long runtimes. LAPS provides a time-efficient solution to interpatient point set comparison as a system whose coordinates are derived from general LA anatomy—not dependent on atrial size, position, or orientation.

Other cohort studies avoid the problems of interpatient image registration by dividing the LA into segments with respect to LA anatomy. Segment definition is not standardized, and they are traditionally defined manually or semiautomatically, which is both time-consuming and prone to user variation, especially if the study is large or the images limit which segments can be reliably identified by a human. LAPS demonstrated the ability to automatically segment the LA wall into validated sizes using user-defined boundary equations (FIGS. 13A-13D). This enables future large cohort studies by allowing time-efficient and standardized analysis of the LA surface.

### III.D. Flexibility for Additional Applications

A flexible data processing tool is useful to meet diverse needs of LA research. The pseudo-cartographic framework of LAPS permits the display of the LA body on a 2D map using dozens of different projections. Texture-mapping showed that the projections maintain the general expected distortion patterns (FIGS. 24A-24C). The PQ projection suit specific needs, but others may be interested in displaying area-preserving maps—perhaps of scarred areas—or distance-preserving maps to display path lines on the LA.

There has been interest in particle-based statistical shape modeling of the LA. LA geometries are typically marked with a few landmark points, and then more particles are added by iteratively solving an energy optimization function to obtain a shape sampling that has correspondence across all geometries. Instead of this expensive operation, LAPS coordinates provide point correspondence by construction. A cohort of LA geometries may be evenly sampled by querying the proper coordinates.

The LAA and PVs do not receive coordinate assignments in LAPS discussed above. LAPS coordinates are calculated independently at each point on a mesh. Other preferred embodiments may not compute coordinates independently at each point but rather determine the coordinates as the solution to boundary equations where coordinate values are initialized at the boundaries defined by the prime meridian, mitral valve center, and posterior center. Such solving methods have been successfully used to compute UAC coordinates and prior art in LV coordinate systems (Cobiveco).

Testing was conducted on patients with known atrial fibrillation and atria dilated beyond healthy ranges. It is feasible that LAPS may perform differently in cohorts where the LA is smaller or less spherical in shape.

### IV. GENERATION AND APPLICATIONS OF STANDARDIZED COORDINATE SYSTEMS

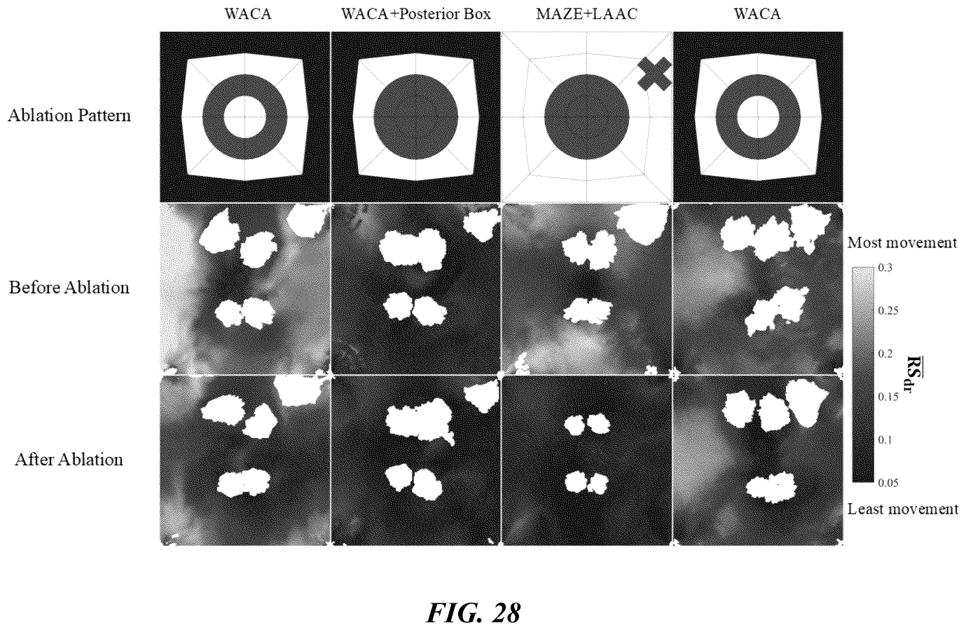

FIG. 29 shows the flowchart of a process for left atrium mapping, according to embodiments of the present disclosure. The process 2900 includes obtaining imaging data of the left atrium of a subject (e.g., a patient) at 2902.

The imaging data may include various types of medical or non-medical imaging data. In some examples, the imaging data may include volumetric medical imaging data such as Computed Tomography (CT), Magnetic Resonance Imaging (MRI), ultrasound, or other three-dimensional (3D) or four-dimensional (4D) imaging modalities. Four-dimensional imaging data may include three spatial dimensions plus a temporal dimension, such as in 4D CT (4DCT) or 4D MRI, where multiple volumes are acquired over time to capture dynamic changes in the target component.

The imaging data may be contrast-enhanced or non-contrast, depending on the application and target component being imaged. For example, contrast-enhanced 4DCT may be used to image cardiac structures with improved delineation of chambers and vessels. In cardiac applications, electrocardiogram (ECG) gating may be used to synchronize image acquisition with specific phases of the cardiac cycle.

In some implementations, the imaging data may be acquired using 3D ultrasound techniques, including transthoracic, transesophageal, or intracardiac echocardiography. For ultrasound applications, the 3D data may be constructed from a series of 2D images that are spatially related to generate volumetric data. Ultrasound elastography may also provide additional functional data for certain applications.

The imaging data may have various resolutions and field-of-view characteristics depending on the imaging modality and acquisition parameters. The resolution of the imaging data may impact, e.g., the level of detail in the resulting surface mesh, though the coordinate system generation techniques described herein are designed to be robust to variations in resolution within practical limits.

The process 2900 includes identifying one or more anatomical features represented in the imaging data at 2904. One or more segmentation techniques are employed to isolate cardiac structures of interest including, e.g., pulmonary veins, the mitral valve, or the like, or a combination thereof. To accomplish this, the process 2900 may include applying a machine learning algorithm, e.g., a model based on a convolutional neural network. The segmentation process may include identifying the volumes of specific cardiac structures and determining boundary planes of the left atrium with an adjacent cardiac structure.

In some embodiments, the segmentation process includes a volumetric approach to identifying cardiac structures. Specifically, the imaging data is processed to extract distinct volumes for a cardiac structure.

Merely by way of example, the boundary plane identification leverages a volume dilation and overlap method. For each identified cardiac structure, the corresponding volume is systematically dilated, expanding its spatial representation. The boundary planes of the left atrium are then precisely determined by identifying the regions where these dilated volumes intersect and overlap.

A resulting boundary planes correspond to an anatomical junction between the left atrium and an adjacent cardiac structure. Examples of these planes include: a pulmonary vein plane located at the junction where a pulmonary vein connects to the left atrium, a mitral valve plane positioned at the junction where the left atrium interfaces with the left ventricle (where the mitral valve is located), a left atrial appendage plane defined at the point where the left atrial appendage connects to the left atrium.

Embodiments of the invention may identify the pulmonary vein planes or mitral valve planes through methods which are not automated or volumetric. The planes might be determined manually, semi-automatically, or automatically—including from machine learning models—directly from medical images or from image segmentations that do not explicitly distinguish the LA body from its surrounding structures.

From these boundary planes, a 3D surface mesh corresponding to the left atrium is generated at 2906. The surface mesh includes nodes that correspond to the outer boundary or surface of the left atrium, including the boundary planes. These nodes correspond to points on the outer surface of the left atrium. A node may be assigned with an anatomical label corresponding to the specific anatomical features or boundary planes of the left atrium. Nodes corresponding to a same anatomical feature or boundary plane may be assigned a same anatomical label. An anatomical feature or boundary plane may be represented by multiple nodes. Merely by way of example, multiple nodes corresponding to a same pulmonary vein plane may be assigned a same anatomical label; these nodes collectively delineate the pulmonary vein plane, on the basis of which a further analysis may be performed (e.g., determining a centroid thereof as described below). The surface mesh may be a triangular surface mesh. The surface mesh may have a pre-defined resolution, e.g., 1 mm, 2 mm, 3 mm, below 5 mm, below 8 mm, etc.

The process 2900 then establishes one or more reference points based on the identified anatomical features. The reference points may adapt to the inherent variability of cardiac anatomy. At 2908, a reference point (or referred to as a first reference point) is determined based on the pulmonary veins. This reference point is located using the centroid of the pulmonary vein planes. The operation may accommodate diverse configurations-whether four separate veins, a common vein on one side, or an atypical vein arrangement. The algorithm adjusts its calculation method to maintain point consistency across different anatomical structures. Whether the subject has four separate pulmonary veins, a common pulmonary vein on one side, or an atypical number of veins, the algorithm adapts its weighting scheme to maintain anatomical consistency. In some embodiments, the process 2900 includes determining a second reference point based on the location of a junction of an anatomical feature, e.g., the mitral valve plane. For example, the second reference point is the centroid of the mitral valve plane. The process 2900 includes determining a reference axis based on the first and/or the second reference points at 2910. For example, the reference axis passes through the first reference point, or the second reference point, or both the first and the second reference points. The reference axis may be a left atrial long axis in this case.

The reference point(s), as well as the reference axis determined based on one or more of these reference points, enables comparison of cardiac structures within a single subject over time and between different subjects. By establishing reference point(s) and/or reference axis that adapt to anatomical variations, the technique provides a framework for tracking changes in left atrial geometry. The method supports analysis of cardiac morphology by creating a standardized approach to mapping left atrial structures.

The process 2900 includes determining lines of longitude and latitude on the surface mesh. At 2912, the lines of longitude are created by defining planes through each target point and the reference axis, finding their intersections with the surface mesh. A prime meridian is established, e.g., using a roof line vector derived from the centroids of the right and left superior pulmonary veins, effectively bisecting the left atrium into left and right halves. Principle component analysis on the 3D coordinate locations of the PVs, for example, can produce a direction vector which optimally bisects the inferior and superior PV centroids. The use of all PVs in the calculation of the roofline stabilizes its orientation across anatomic variations. There may be other definitions for the roof line vector, such as methods that compute a vector passing between all PV centroids rather than connecting any two. As another example, a roof line may be a vector determined based on at least one set of contralateral or ipsilateral pulmonary veins, or based on at least one set of two or more inferior veins or superior ones.

At 2914, lines of latitude are generated as isolines of equal normalized distance along the longitude lines. At 2916, each point on the surface mesh is then assigned unique coordinate values, comprising a latitude value and a longitude value. For a specific point, the latitude value represents the distance from a reference point (e.g., the first or the second reference point) along the line of longitude that traverse the point, and the longitude value indicates the distance from the prime meridian. The distance may be normalized to a value, e.g., a value between 0 and 1. This normalization transforms variable cardiac geometries into a comparable coordinate space. By mapping each point using a consistent method, the technique creates a framework that can represent left atrial structures independently of individual anatomical differences.

The process 2900 includes generating a two-dimensional map of the left atrium at 2918. This map provides a standardized, geometrically consistent representation that can be further enhanced by mapping various physiological parameters.

In various implementations, one or more physiological parameters associated with the left atrium may be mapped onto the two-dimensional representation to enable visualization, analysis, and comparison of these parameters across different regions of the left atrium or between different subjects. The standardized coordinate system facilitates consistent mapping of these parameters to specific locations on the left atrial surface, allowing for meaningful comparison despite variations in left atrial size, shape, or orientation.

Examples of physiological parameters that may be mapped include electrophysiological measurements, structural characteristics, mechanical properties, and functional metrics. In some examples, the physiological parameters may include:

Electrophysiological parameters (or electrical properties) may include voltage measurements from electroanatomic mapping, activation timing data, conduction velocity vectors, complex fractionated atrial electrogram (CFAE) scores, dominant frequency analyses, rotor locations, or regions of electrical silence. These electrical parameters may be particularly relevant for identifying arrhythmogenic substrate or planning ablation procedures.

Structural parameters may include wall thickness measurements, fibrosis or scar quantification derived from late gadolinium enhancement MRI (LGE-MRI), computed tomography (CT) attenuation values, ultrasound-derived tissue characterization metrics, or microstructural parameters such as fiber orientation. Structural parameters may help identify regions of pathological remodeling or substrates for arrhythmias.

Mechanical parameters may include strain values (e.g., longitudinal strain, circumferential strain, radial strain), strain rate, displacement, velocity, or other deformation metrics derived from imaging data. Regional mechanical dysfunction may serve as a surrogate for underlying disease processes or predict response to interventions.

In various implementations, the physiological parameters may be represented on the two-dimensional map using color coding, contour lines, vector fields, numerical annotations, or other visualization techniques. For example, a color scale may be applied to represent the magnitude of a scalar parameter such as voltage amplitude or wall thickness, while arrows or streamlines may be used to represent vector quantities such as conduction velocity or strain direction.

The two-dimensional representation allows simultaneous visualization of multiple physiological parameters, enabling correlation analyses between different metrics across different regions of the left atrium for clinical management for the subject or a subject group (or referred to as cohort group) that includes at least another subject other than the subject. For instance, regions of low voltage may be overlaid with areas of reduced strain to identify relationships between electrical and mechanical dysfunction.

The two-dimensional parameterization allows simultaneous visualization and spatial co-registration of multiple physiological parameters in three-dimensional space. For instance, a 3D representation of wall thickness may be co-registered to a 3D representation of electrical properties so that the amount of energy applied in an ablation procedure can be calibrated to the needs of the local tissue.

In some implementations, the mapping of physiological parameters may include temporal information, such as changes in parameter values over the cardiac cycle or across different phases of respiration. This temporal dimension may be represented through animation, separate frames for different time points, or summary statistics derived from the temporal data.

The standardized mapping of physiological parameters facilitates quantitative comparison within predefined anatomical regions or custom-defined regions of interest to allow clinical management for the subject or a subject group (or referred to as cohort group) that includes at least another subject other than the subject. Statistical measures such as mean, median, standard deviation, or percentile values may be calculated for each region, enabling regional comparisons and the identification of outliers or abnormal regions. Examples of clinical management include ablation procedure planning, intervention energy calibration, treatment response assessment, or longitudinal disease progression monitoring.

For the same subject, the process enables tracking morphological and/or functional changes over time by generating comparative maps of the left atrium of the subject. For a subject group, the process supports generating statistical atlases that characterize left atrial morphology and/or function across individual patients. These approaches support personalized medical decision-making and comparative assessment, providing insights into individual patient progression and population-level cardiac characteristics. The process further enables clinical management of a second subject by leveraging information derived from the subject or the subject group to which both the different subject and the original subject belong. By establishing a standardized coordinate framework and statistical reference, a user (e.g., a clinician, a researcher) can use the mapped physiological parameters to inform diagnostic, prognostic, and therapeutic strategies for a subject who shares similar medical condition, and/or morphological or functional characteristics with the original subject or subject group.

This technique represents advancement in medical imaging and computational anatomy, offering precision in left atrium mapping while maintaining the flexibility to accommodate individual anatomical variations. The generation of the standardized coordinate system represents a computationally efficient approach to mapping left atrial anatomy. By leveraging anatomical landmarks and employing straightforward geometric calculations, the technology creates a coordinate framework with minimal computational overhead.

The process begins with robust imaging data acquisition, accommodating various modalities including, for example, CT, MRI, and ultrasound. Advanced segmentation techniques, powered by machine learning algorithms, can be employed to automatically and precisely identify cardiac structures. A volume dilation and overlap method enables accurate boundary plane determination, addressing the inherent variability of cardiac anatomy.

Reference point calculation demonstrates the adaptability of the process. By dynamically adjusting to different pulmonary vein configurations, the algorithm maintains anatomical consistency. The resulting coordinate system provides a standardized framework for comparing cardiac structures across subjects and over time.

Lines of longitude and latitude are generated through direct geometric intersections on the surface mesh. A point's coordinate values are calculated using normalized distance measurements along these lines. This approach eliminates the need for complex interpolation or advanced optimization algorithms.

The coordinate generation process uses direct geometric intersections and normalized distance measurements, eliminating complex interpolation requirements. This approach enables rapid mapping of left atrial geometries, supporting large-scale medical imaging research with computational efficiency.

The technique transforms complex cardiac anatomy into a systematic, mappable framework, offering a powerful tool for investigating cardiac morphology.

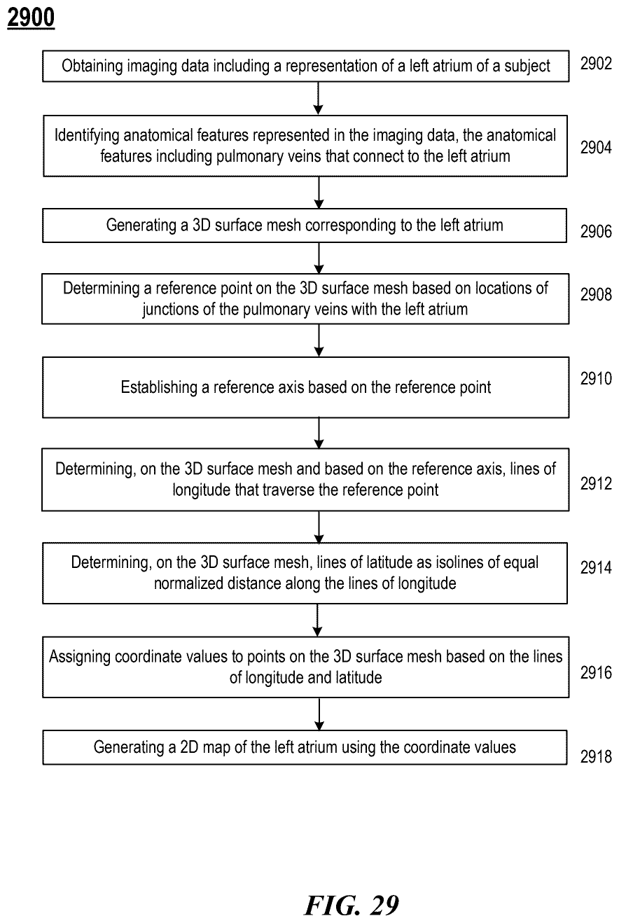

FIG. 30 shows the flowchart of a process for mapping a three-dimensional target component, according to embodiments of the present disclosure.

The process 3000 includes obtaining imaging data that includes a representation of a 3D target component at 3002. The techniques described herein are applicable to various types of 3D or 4D imaging data from which surface representations of 3D target components can be derived. This operation parallels the imaging data acquisition in the left atrium mapping process 2900, supporting various medical imaging modalities such as CT, MRI, and ultrasound.

The imaging data may include various types of medical or non-medical imaging data. In some examples, the imaging data may include volumetric medical imaging data such as CT, MRI, ultrasound, or other 3D or 4D imaging modalities. Four-dimensional imaging data may include three spatial dimensions plus a temporal dimension, such as in 4DCT or 4D MRI, where multiple volumes are acquired over time to capture dynamic changes in the target component.

The imaging data may be contrast-enhanced or non-contrast, depending on the application and target component being imaged. For example, contrast-enhanced 4DCT may be used to image cardiac structures with improved delineation of chambers and vessels.

In some implementations, the imaging data may be acquired using 3D ultrasound techniques, including transthoracic, transesophageal, or intracardiac echocardiography. For ultrasound applications, the 3D data may be constructed from a series of 2D images that are spatially related to generate volumetric data. Ultrasound elastography may also provide additional functional data for certain applications.

The imaging data may have various resolutions and field-of-view characteristics depending on the imaging modality and acquisition parameters. The resolution of the imaging data may impact the level of detail in the resulting 3D surface mesh, though the coordinate system generation techniques described herein are designed to be robust to variations in resolution within practical limits.

At 3004, the process 3000 includes identifying, by segmenting the imaging data, the target component and feature regions associated with the target component. Machine learning algorithms, e.g., convolutional neural network models, may be applied to perform the segmentation. The segmentation may include determining a boundary plane between the target component and a feature region adjacent to the target component.

At 3006, the process 3000 includes generating a 3D surface mesh. In some embodiments, the 3D surface mesh is a triangular mesh, including nodes corresponding to or representing an outer boundary or surface of the target component.

A node within the mesh can be assigned an anatomical or feature label that indicates the specific region or structural characteristic of the target component represented by that point. For example, a node representing a portion of a boundary plane between the target component and a feature region A may be assigned a label indicating the association with the feature region A.

The 3D surface mesh demonstrates adaptability to the unique geometric characteristics of the target component. In some embodiments, it supports variable mesh resolutions-ranging from fine-grained representations (such as 1 mm or 2 mm resolution) to more generalized configurations. This flexibility allows the mesh to capture structural details at different levels of precision.

The mesh's ability to adapt to the component's geometry ensures that complex structural variations can be accurately represented. Whether dealing with regular or irregular geometric configurations, the triangular mesh provides a robust framework for spatial mapping and analysis.

Multiple nodes may collectively represent a single anatomical feature or boundary plane. For instance, nodes corresponding to a specific feature region (e.g., a pulmonary vein plane, the mitral valve plane in the exemplary implementation of left atrium mapping) can be assigned a consistent label, enabling collective analysis of that region.

At 3008, the process 3000 includes establishing a reference axis. By identifying and connecting one or more reference points—e.g., those derived from feature anatomical junctions—the method creates a consistent spatial reference. This axis serves as more than a geometric construct; it enables comparisons across different instances of the component, providing a standardized approach to structural analysis. For example, the process 3000 includes determining a reference point (or referred to as a first reference point) based on at least one of the feature regions; and determining the reference axis based on the reference point. In some embodiments, the process 3000 includes identifying a second reference point based on at least a second feature region, and determining the reference axis based on the first reference point and the second reference point. In some embodiments, the second feature region may be part of the identified feature regions. The second feature region may be different from at least one of the feature region used to determine the first reference point.

At 3010, the process 3000 includes determining lines of longitude. For each target location or point on the 3D surface mesh, a two-dimensional plane is defined using the reference axis. An intersection of the 2D plane with the 3D surface mesh may be determined as a line of longitude that traverses the target point. One of lines of longitude may be designated as a prime meridian.

At 3012, the process 3000 includes determining lines of latitude. In some embodiments, these lines represent paths of equal relative position, traversing the longitude lines and creating a comprehensive coordinate network. They transform the geometric space into a normalized, comparable representation.

At 3014, the process 3000 includes assigning coordinate values to points on the 3D surface mesh based on the lines of longitude and latitude. Coordinate assignment transforms the geometric framework into a quantifiable system. Each point receives unique coordinate values—a latitude and longitude—normalized between zero and one. The latitude value traces a point's distance from a reference point (e.g., the first or the second reference point that defines the reference axis), while the longitude value measures its position relative to the prime meridian.

At block 3016, the process 3000 includes generating a two-dimensional representation by distilling the three-dimensional anatomical structure into a standardized map. This transformation serves as a powerful analytical tool, facilitating detailed examination of structural variations and the identification of underlying patterns. In some embodiments, the resulting map provides a standardized and geometrically consistent framework that can be further enriched by overlaying various physiological parameters. This is analogous to the approach described with reference to FIG. 29, which is applicable here but not repeated for brevity.

The process 3000 provides an approach to geometric translation, bridging complex structural data and systematic understanding. By providing a versatile, computationally efficient mapping technique, the process offers a robust method for transforming raw data into meaningful, comparable representations, facilitating investigations of intricate three-dimensional components.

In some embodiments, the present technology provides an analytical tool for comprehensively mapping biological structures that undergo non-rigid volume changes, where dimensional transformations across different regions are nonuniform and not constrained by rigid geometric principles. The technique addresses the challenge of capturing and comparing complex morphological variations in biological components that exhibit dynamic, spatially heterogeneous deformations.

By establishing a standardized coordinate framework, the technique enables precise tracking and comparison of structural changes across regions with independently varying geometries. The two-dimensional representation transforms complex three-dimensional data into an interpretable format that preserves local spatial relationships and regional variation, even as the overall structure undergoes significant non-uniform deformation.

The coordinate mapping approach supports comparative analysis across temporal and population dimensions. For the same subject, the technique enables tracking physiological parameters over time, capturing subtle changes in regional characteristics across different imaging sessions or physiological states. Simultaneously, the technique supports comparative analysis across different subjects by generating statistical atlases that characterize morphological and functional variations within a subject group or population.

This approach allows simultaneous visualization and spatial co-registration of multiple physiological parameters, accounting for nonlinear and region-specific dimensional changes. By generating coordinate values that normalize regional differences, the technique enables meaningful comparisons within a single subject's physiological progression and across different subjects sharing similar characteristics.

The method provides a consistent geometric framework that can accommodate substantial local variations while maintaining a standardized comparative approach. This capability is critical for analyzing biological structures that experience complex morphological transformations, such as soft tissues, organs during functional states, or dynamic physiological systems.

Through its ability to map non-rigidly deforming biological components and preserve regional geometric variations, the technique offers a tool for quantitative analysis of complex morphological changes. The standardized coordinate framework transforms the challenge of comparing nonuniform transformations into a systematic, interpretable methodology.

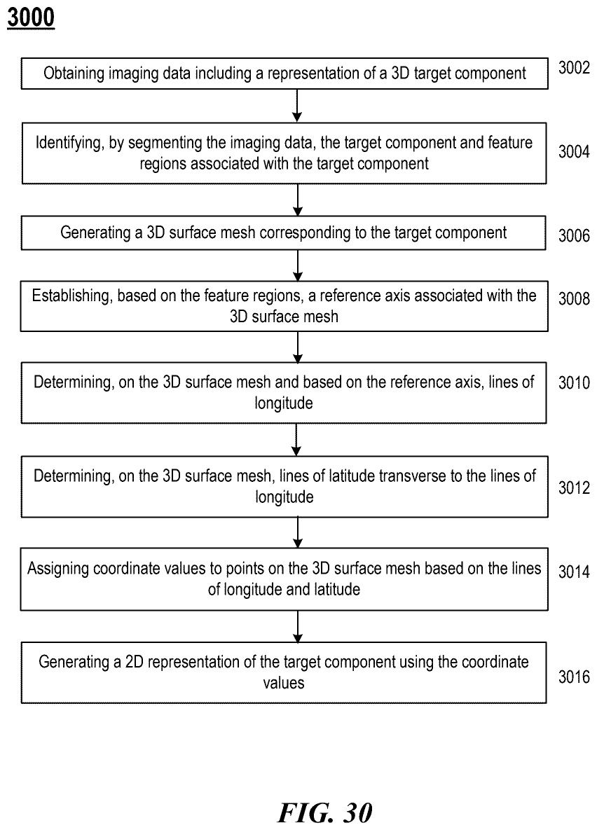

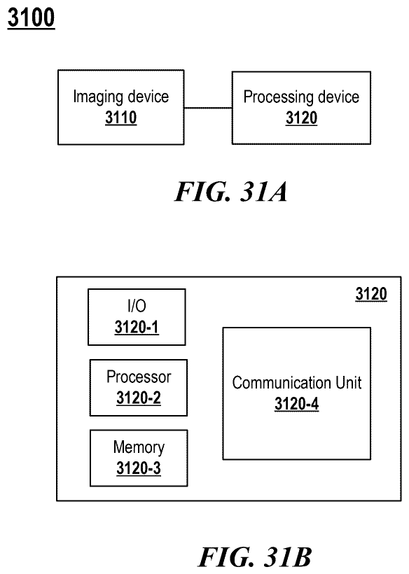

FIG. 31A shows a block diagram of an example system, according to embodiments of the present technology. In some embodiments, the processing device 3120 may be in communication with one or more components of the system 3100. For example, the processing device 3120 may be in communication with the imaging device 3110 and configured to process the imaging data obtained by the imaging device 3110 following the process 2900 or 3000. Examples of the imaging device 3110 include a CT scanner, an MRI scanner, a 3D ultrasound system, etc.

FIG. 31B shows a block diagram of an exemplary processing device, according to embodiments of the present technology. In various implementations, the processing device 3120 is embodied on one or more personal computing devices, e.g., including a desktop or laptop computer, one or more computing devices in a computer system or communication network accessible via the Internet (referred to as “the cloud”) including servers and/or databases in the cloud, and/or one or more mobile computing devices, such as a smartphone, tablet, or wearable computer device including a smartwatch or smartglasses. The processing device 3120 includes a processor to process data, and memory in communication with the processor to store and/or buffer data. For example, the processor can include a central processing unit (CPU) or a microcontroller unit (MCU). In some implementations, the processor can include a field-programmable gate-array (FPGA) or a graphics processing unit (GPU). For example, the memory can include and store processor-executable code, which when executed by the processor, configures the data processing device 3120 to perform various operations, e.g., such as receiving information, commands, and/or data, processing information and data, such as from the system 3100, and transmitting or providing processed information/data to another device, such as an external display. To support various functions of the data processing device 3120, the memory can store information and data, such as instructions, software, values, images, and other data processed or referenced by the processor. For example, various types of Random Access Memory (RAM) devices, Read Only Memory (ROM) devices, Flash Memory devices, and other suitable storage media can be used to implement storage functions of the memory. In some implementations, the data processing device 3120 includes an input/output (I/O) unit 3120-1 to interface the processor 3120-2 and/or memory 3120-3 to other modules, units or devices. In some embodiments, such as for mobile computing devices, the data processing device 3120 includes a communications unit to facilitate data exchange via a wired or wireless connection, e.g., such as a transmitter (Tx) or a transmitter/receiver (Tx/Rx) unit. For example, in such embodiments, the I/O unit 3120-1 can interface the processor and memory with the wireless communications unit, e.g., to utilize various types of wireless interfaces compatible with typical data communication standards, which can be used in communications of the processing device 3120 with other devices, e.g., such as between the one or more computers in the cloud and the user device. The data communication standards include, but are not limited to, Bluetooth, Bluetooth low energy (BLE), Zigbee, IEEE 802.11, Wireless Local Area Network (WLAN), Wireless Personal Area Network (WPAN), Wireless Wide Area Network (WWAN), WiMAX, IEEE 802.16 (Worldwide Interoperability for Microwave Access (WiMAX)), 3G/4G/LTE/5G cellular communication methods, and parallel interfaces. In some implementations, the data processing device 3120 can interface with other devices using a wired connection via the I/O unit. The data processing device 3120 can also interface with other external interfaces, sources of data storage, and/or visual or audio display devices, etc. to retrieve and transfer data and information that can be processed by the processor, stored in the memory, or exhibited on an output unit of a display device or an external device.

The following examples are illustrative of several embodiments of the present technology:

Solution 1. A computer-implemented method for left atrium mapping, comprising: obtaining imaging data including a representation of a left atrium of a subject; identifying anatomical features represented in the imaging data, the anatomical features including pulmonary veins that connect to the left atrium; generating a three-dimensional (3D) surface mesh corresponding to the left atrium; determining a reference point on the 3D surface mesh based on locations of junctions of the pulmonary veins with the left atrium; establishing a reference axis based on the reference point; determining, on 3D the surface mesh and based on the reference axis, lines of longitude that traverse the reference point; determining, on the 3D surface mesh, lines of latitude as isolines of equal normalized distance along the lines of longitude; assigning coordinate values to points on the 3D surface mesh based on the lines of longitude and latitude; and generating a two-dimensional (2D) map of the left atrium using the coordinate values. The 2D map enables a visualization or comparison of a physiological parameter across different regions of the left atrium for clinical management for the subject or a subject group (or referred to as cohort group) that includes at least another subject other than the subject.

Solution 2. The method of any one or more of Solution 1 or other solutions disclosed herein, wherein determining lines of longitude comprises: for each of a plurality of target points on the 3D surface mesh: defining a 2D plane defined by the target point and the reference axis; and determining, on the 3D surface mesh, an intersection of the plane with the 3D surface mesh as a line of longitude through the target point.

Solution 3. The method of any one or more of Solution 1 or other solutions disclosed herein, wherein: the lines of longitude comprise a prime meridian; and the determining lines of longitude comprises determining the prime meridian by: defining a roof line vector based on locations of junctions of two or more of the pulmonary veins with the left atrium; determining a direction reference vector based on the reference axis and the roof line vector; and determining an intersection of the 3D surface mesh with a plane formed by the reference axis and the direction reference vector. The prime meridian bisects the left atrium into left and right halves.

Solution 4. The method of any one or more of Solution 1 or other solutions disclosed herein, wherein the anatomical features include a mitral valve, and the method comprises determining a second reference point on the 3D surface mesh based on a location of a junction of the mitral valve with the left atrium. The reference axis is established based on the reference point and the second reference point. In some embodiments, each of at least some of the lines of longitude connects both the reference point (or referred to as the first reference point) and the second reference points.

Solution 5. The method of any one or more of Solution 1 or other solutions disclosed herein, wherein the coordinate values of the points comprise a latitude value and a longitude value for each of the points.

Solution 6. The method of any one or more of Solution 5 or other solutions disclosed herein, wherein the latitude value of one of the points relates to a distance between the point and the reference point along the line of longitude through the point.

Solution 7. The method of any one or more of Solution 5 or other solutions disclosed herein, wherein the longitude value of one of the points relates to a distance between the line of longitude through the point and a prime meridian.

Solution 8. The method of any one or more of Solution 1 or other solutions disclosed herein, wherein the reference axis is a left atrial long axis.

Solution 9. The method of any one or more of Solution 1 or other solutions disclosed herein, wherein: the identifying the anatomical features represented in the imaging data comprises: segmenting the imaging data to identify cardiac structures including at least one of the left atrium, a pulmonary vein, or the mitral valve; and determining boundary planes of the left atrium based on the identified cardiac structures; and the generating the 3D surface mesh is based on the boundary planes of the left atrium.

Solution 10. The method of any one or more of Solution 9 or other solutions disclosed herein, wherein the segmenting the imaging data comprises applying a machine learning-based segmentation algorithm on the imaging data.

Solution 11. The method of any one or more of Solution 10 or other solutions disclosed herein, wherein the machine learning-based segmentation algorithm comprises a convolutional neural network architecture.

Solution 12. The method of any one or more of Solution 9 or other solutions disclosed herein, wherein: the segmenting the imaging data comprises identifying a volume for each of the identified cardiac structures; and the determining the boundary planes of the left atrium based on the identified cardiac structures comprises: dilating the identified volumes corresponding to each of the identified cardiac structures; and identifying the boundary planes of the left atrium based on regions where the dilated volumes overlap.

Solution 13. The method of any one or more of Solution 9 or other solutions disclosed herein, wherein the boundary planes include at least one of: a pulmonary vein plane at a junction where a pulmonary vein connects to the left atrium; a mitral valve plane at a junction where the left atrium connects to a left ventricle (where the mitral valve is located); or a left atrial appendage plane at a junction where a left atrial appendage connects to the left atrium.

Solution 14. The method of any one or more of Solution 13 or other solutions disclosed herein, wherein the reference point is determined based on a centroid of pulmonary vein planes corresponding to two or more of the pulmonary veins.

Solution 15. The method of any one or more of Solution 13 or other solutions disclosed herein, wherein the second reference point is determined based on a centroid of the mitral valve plane.

Solution 16. The method of any one or more of Solution 9 or other solutions disclosed herein, wherein the 3D surface mesh comprises a plurality of nodes that correspond to the boundary planes and have anatomical labels corresponding to the respective anatomical features.

Solution 17. The method of any one or more of Solution 1 or other solutions disclosed herein, wherein the determining the reference point comprises: identifying a pattern of the pulmonary veins, the pattern including at least one of: four separate pulmonary veins, a common pulmonary vein on at least one side, or an atypical number of pulmonary veins.

Solution 18. The method of any one or more of Solution 17 or other solutions disclosed herein, wherein the determining the reference point comprises: adapting a weighting scheme based on the identified pattern of the pulmonary veins to maintain anatomical consistency of the first reference point across different pulmonary vein configurations.

Solution 19. The method of any one or more of Solution 1 or other solutions disclosed herein, comprising: mapping values of the physiological parameter of the left atrium onto the 2D map. The physiological parameter may include at least one of: wall thickness, strain, tissue scarring, perfusion, or an electrophysiological parameter. The clinical management may include at least one of: ablation procedure planning, intervention energy calibration, treatment response assessment, or longitudinal disease progression monitoring.

Solution 20. A computer-implemented method, comprising: obtaining imaging data including a representation of a three-dimensional (3D) target component; identifying, by segmenting the imaging data, the target component and feature regions associated with the target component; generating a 3D surface mesh corresponding to the target component; establishing, based on the feature regions, a reference axis associated with the 3D surface mesh; determining, on the 3D surface mesh and based on the reference axis, lines of longitude; determining, on the 3D surface mesh, lines of latitude transverse to the lines of longitude; assigning coordinate values to points on the 3D surface mesh based on the lines of longitude and latitude; and generating a two-dimensional (2D) representation of the target component using the coordinate values.

Solution 21. The method of any one or more of Solution 20 or other solutions disclosed herein, wherein the establishing the reference axis comprises: identifying a reference point based on at least one of the feature regions; and determining the reference axis based on the reference point. In some embodiments, the method may include identifying a second reference point based on at least a second feature region, and the reference axis is determined based on the reference point (or referred to as a first reference point) and the second reference point. The second feature region may be part of the identified feature regions. The second feature region may be different from at least one of the feature region used to determine the first reference point.

Solution 22. The method of any one or more of Solution 20 or other solutions disclosed herein, wherein the determining the lines of longitude comprises: for a target location on the 3D surface mesh, determining a 2D plane based on the reference axis and the target location; and determine, on the 3D surface mesh, an intersection of the plane with the 3D surface mesh as a line of longitude through the target point.

Solution 23. The method of any one or more of Solution 20 or other solutions disclosed herein, wherein the lines of latitude represent paths of equal relative position along the lines of longitude.

Solution 24. The method of any one or more of Solution 20 or other solutions disclosed herein, wherein the coordinate values of the points comprise a latitude value and a longitude value for each of the points.

Solution 25. The method of any one or more of Solution 24 or other solutions disclosed herein, wherein the latitude value of one of the points relates to a distance between the point and the first or second reference point along the line of longitude through the point.

Solution 26. The method of any one or more of Solution 24 or other solutions disclosed herein, wherein: the lines of longitude comprise a prime meridian; and the longitude value of one of the points relates to a distance between the line of longitude through the point and the prime meridian.

Solution 27. A system, comprising: at least one processor; and memory with instructions stored thereon, wherein the instructions upon execution by the at least one processor, cause the at least one processor to perform operations of the method of any one or more of the solutions disclosed herein.

Solution 28. One or more non-transitory computer readable program storage media having code stored thereon, the code, when executed by a processor, causing the processor to implement a method of any one or more of the solutions disclosed herein.

Implementations of the subject matter and the functional operations described in this patent document can be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing unit” or “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

The term “about,” as used herein when referring to a measurable value such as an amount or concentration and the like, is meant to encompass variations of 20%, 10%, 5%, 1%, 0.5%, or even 0.1% of the specified amount.

It is intended that the specification, together with the drawings, be considered exemplary only, where exemplary means an example. As used herein, the singular forms “a”, “an” and “the” are intended to include the plural forms as well, unless the context clearly indicates otherwise. Additionally, the use of “or” is intended to include “and/or”, unless the context clearly indicates otherwise.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

1. A computer-implemented method, comprising:
obtaining imaging data including a representation of a left atrium of a subject;
identifying anatomical features represented in the imaging data, the anatomical features including pulmonary veins that connect to the left atrium;
generating a three-dimensional (3D) surface mesh corresponding to the left atrium;
determining a reference point on the 3D surface mesh based on locations of junctions of the pulmonary veins with the left atrium;
establishing a reference axis based on the reference point;
determining, on the 3D surface mesh and based on the reference axis, lines of longitude that traverse the reference point;
determining, on the 3D surface mesh, lines of latitude as isolines of equal normalized distance along the lines of longitude;
assigning coordinate values to points on the 3D surface mesh based on the lines of longitude and latitude; and
generating a two-dimensional (2D) map of the left atrium using the coordinate values, wherein the 2D map enables a visualization or comparison of a physiological parameter across different regions of the left atrium for clinical management for the subject or a subject group that includes at least another subject other than the subject.

2. The method of claim 1, wherein determining lines of longitude comprises:
for each of a plurality of target points on the 3D surface mesh:
defining a 2D plane defined by the target point and the reference axis; and
determining, on the 3D surface mesh, an intersection of the plane with the 3D surface mesh as a line of longitude through the target point.

3. The method of claim 1, wherein:
the lines of longitude comprise a prime meridian; and
the determining lines of longitude comprises determining the prime meridian by:
defining a roof line vector based on locations of junctions of two or more of the pulmonary veins with the left atrium;
determining a direction reference vector based on the reference axis and the roof line vector; and
determining an intersection of the 3D surface mesh with a plane formed by the reference axis and the direction reference vector.

4. The method of claim 1, wherein:
the anatomical features include a mitral valve, and
the method comprises determining a second reference point on the 3D surface mesh based on a location of a junction of the mitral valve with the left atrium, wherein the reference axis is established based on the reference point and the second reference point.

5. The method of claim 1, wherein the coordinate values of the points comprise a latitude value and a longitude value for each of the points.

6. The method of claim 5, wherein the latitude value of one of the points relates to a distance between the point and the reference point along the line of longitude through the point.

7. The method of claim 5, wherein the longitude value of one of the points relates to a distance between the line of longitude through the point and a prime meridian.

8. The method of claim 1, wherein the reference axis is a left atrial long axis.

9. The method of claim 1, wherein:
the identifying the anatomical features represented in the imaging data comprises:
segmenting the imaging data to identify cardiac structures including at least one of the left atrium, a pulmonary vein, or a mitral valve; and
determining boundary planes of the left atrium based on the identified cardiac structures; and
the generating the 3D surface mesh is based on the boundary planes of the left atrium.

10. The method of claim 9, wherein the segmenting the imaging data comprises applying a machine learning-based segmentation algorithm on the imaging data.

11. The method of claim 9, wherein the boundary planes include at least one of:
a pulmonary vein plane at a junction where a pulmonary vein connects to the left atrium;
a mitral valve plane at a junction where the left atrium connects to a left ventricle; or
a left atrial appendage plane at a junction where a left atrial appendage connects to the left atrium.

12. The method of claim 11, wherein the reference point is determined based on a centroid of pulmonary vein planes corresponding to two or more of the pulmonary veins.

13. The method of claim 1, comprising:
mapping values of the physiological parameter of the left atrium onto the 2D map.

14. The method of claim 1, wherein the physiological parameter comprises at least one of: wall thickness, electrical properties, strain, tissue scarring, perfusion, or electrophysiological characteristics.

15. The method of claim 1, wherein the clinical management comprises at least one of:
ablation procedure planning, intervention energy calibration, treatment response assessment, or longitudinal disease progression monitoring.

16. A computer-implemented method, comprising:
obtaining imaging data including a representation of a three-dimensional (3D) target component;
identifying, by segmenting the imaging data, the target component and feature regions associated with the target component;
generating a 3D surface mesh corresponding to the target component;
establishing, based on the feature regions, a reference axis associated with the 3D surface mesh;
determining, on the 3D surface mesh and based on the reference axis, lines of longitude;
determining, on the 3D surface mesh, lines of latitude transverse to the lines of longitude;
assigning coordinate values to points on the 3D surface mesh based on the lines of longitude and latitude; and
generating a two-dimensional (2D) representation of the target component using the coordinate values.

17. The method of claim 16, wherein the establishing the reference axis comprises:
identifying a reference point based on at least one of the feature regions; and
determining the reference axis based on the reference point.

18. The method of claim 16, wherein the determining the lines of longitude comprises:
for a target location on the surface mesh, determining a 2D plane based on the reference axis and the target location; and
determine, on the surface mesh, an intersection of the plane with the surface mesh as a line of longitude through the target point.

19. The method of claim 16, wherein the coordinate values of the points comprise a latitude value and a longitude value for each of the points.

20. A system, comprising:
at least one processor; and
memory with instructions stored thereon, wherein the instructions upon execution by the at least one processor, cause the at least one processor to perform operations including:
obtaining imaging data including a representation of a three-dimensional (3D) target component;
identifying, by segmenting the imaging data, the target component and feature regions associated with the target component;
generating a 3D surface mesh corresponding to the target component;
establishing, based on the feature regions, a reference axis associated with the 3D surface mesh;
determining, on the 3D surface mesh and based on the reference axis, lines of longitude;
determining, on the 3D surface mesh, lines of latitude transverse to the lines of longitude;
assigning coordinate values to points on the 3D surface mesh based on the lines of longitude and latitude; and
generating a two-dimensional (2D) representation of the target component using the coordinate values.
